SEQUENCE LISTING (4 sequences)

SEQ ID NO: 1 - DNA, 2003 bp, Homo sapiens
  CDS (1)...(1245)
  misc_feature (1)...(2003): n = A,T,C or G
atgactctggctctgagtcacctaatgataccgatacctactgctgtgggtaggtacaccgcaggattctaccgaagtctgtgcctggccctggccctgctcgtggccgtgacggtgttccaacgcagtctcacccctggtcagtttctgcaggagcctccgccacccaccctggagccacagaaggcccagaagccaaatggacagctggtgaaccccaacaacttctggaagaacccgaaagatgtggctgcgcccacgcccatggcctctcaggggccccaggcctgggacgtgaccaccactaactgctcagccaatatcaacttgacccaccagccctggttccaggtcctggagccgcagttccggcagtttctcttctaccgccactgccgctacttccccatgctgctgaaccacccggagaagtgcaggggcgatgtctacctgctggtggttgtcaagtcggtcatcacgcagcacgaccgccgcgaggccatccgccagacctggggccgcgagcggcagtccgcgggtgggggccgaggcgccgtgcgcaccctcttcctgctgggcacggcctccaagcaggaggagcgcacgcactaccagcagctgctggcctacgaagaccgcctctacggcgacatcctgcagtggggctttctcgacaccttcttcaacctgaccctcaaggagatccacttcctcaagtggctggacatctactgcccccacatccccttcattttcaaaggcgacgatgacgtcttcgtcaacctcaccaacctgctagaatttgctgctgaccggcagccacaggaaaacctgttcgtgggcgatgtcctgcagcacgctcggcccattcgcaggaaagacaacaaatactacatcccgggggccctgtacgcaaggccagctagtcccgtatcaggcggcggtggcttcctcaatggccgcagcctggcccggcgcctgcaccatgcctgcgacaccctggagctctacccgatcgacgacgtctttctgggcatgtgcctggaggtgctgggcgtgcagcccacggcccacgagggcttcaagactttcggcatctcccggaaccgcaacagccgcatgaacaaggagccgtgctttttccgcgccatgctcgtggtgcacaagctgctgccccctgagctgctcgccatgtgggggctggtgcacagcaatctcacctgctcccgcaagctccaggtgctctgaccccagccgggctactaggacaggccagggcacttgctcctgagcccccatggtattggggctggagccacagtgcccaggcctagcctttggtccccaaggggaggtggagggttgaggcctacgtgccactgggtgtggtggggtgcaggtagccagaaagggacctccctgtgtggataattctaggaaactgaggcccaggaacggttggagctgcccagtctggaggccctctctgaggagcgaggcgccaggccctggcagccctcctgacctgggtccgttgctggccccctcagatgtggtgggaggtcctggtgacctctggaggaacgctgtgctcaggtacctgggctaggcctggcctgatgggtctgtggccgcccctcgtcttcacagggaagagtcttctgtgaaatgcctcagtctccccagaggccgggcggccctggcaggagaaactcaaccctgtgcgggctcacaggcaccccccagtccacaccctggtctcctgggagagagggcccagccggctctccgcagccccaggcctgcctggagacgggccgcctctgccacagggcctccactcctggctgtgtcctgtaaggtctggaagggcgaccgctctgactacctcagcgcccctcagaatctccctggggctgcagccntaccccaccccgacacagggcagaagannagcnntcctggcccccgaagtcccagagtgttgaccccaacccaggcaa

SEQ ID NO: 2 - protein, 415 aa, Homo sapiens
MTLALSHLMIPIPTAVGRYTAGFYRSLCLALALLVAVTVFQRSLTPGQFLQEPPPPTLEPQKAQKPNGQLVNPNNFWKNPKDVAAPTPMASQGPQAWDVTTTNCSANINLTHQPWFQVLEPQFRQFLFYRHCRYFPMLLNHPEKCRGDVYLLVVVKSVITQHDRREAIRQTWGRERQSAGGGRGAVRTLFLLGTASKQEERTHYQQLLAYEDRLYGDILQWGFLDTFFNLTLKEIHFLKWLDIYCPHIPFIFKGDDDVFVNLTNLLEFAADRQPQENLFVGDVLQHARPIRRKDNKYYIPGALYARPASPVSGGGGFLNGRSLARRLHHACDTLELYPIDDVFLGMCLEVLGVQPTAHEGFKTFGISRNRNSRMNKEPCFFRAMLVVHKLLPPELLAMWGLVHSNLTCSRKLQVL

SEQ ID NO: 3 - DNA, 1245 bp, Artificial Sequence
  degenerate polynucleotide sequence
atgacnytngcnytnwsncayytnatgathccnathccnacngcngtnggnmgntayacngcnggnttytaymgnwsnytntgyytngcnytngcnytnytngtngcngtnacngtnttycarmgnwsnytnacnccnggncarttyytncargarccnccnccnccnacnytngarccncaraargcncaraarccnaayggncarytngtnaayccnaayaayttytggaaraayccnaargaygtngcngcnccnacnccnatggcnwsncarggnccncargcntgggaygtnacnacnacnaaytgywsngcnaayathaayytnacncaycarccntggttycargtnytngarccncarttymgncarttyytnttytaymgncaytgymgntayttyccnatgytnytnaaycayccngaraartgymgnggngaygtntayytnytngtngtngtnaarwsngtnathacncarcaygaymgnmgngargcnathmgncaracntggggnmgngarmgncarwsngcnggnggnggnmgnggngcngtnmgnacnytnttyytnytnggnacngcnwsnaarcargargarmgnacncaytaycarcarytnytngcntaygargaymgnytntayggngayathytncartggggnttyytngayacnttyttyaayytnacnytnaargarathcayttyytnaartggytngayathtaytgyccncayathccnttyathttyaarggngaygaygaygtnttygtnaayytnacnaayytnytngarttygcngcngaymgncarccncargaraayytnttygtnggngaygtnytncarcaygcnmgnccnathmgnmgnaargayaayaartaytayathccnggngcnytntaygcnmgnccngcnwsnccngtnwsnggnggnggnggnttyytnaayggnmgnwsnytngcnmgnmgnytncaycaygcntgygayacnytngarytntayccnathgaygaygtnttyytnggnatgtgyytngargtnytnggngtncarccnacngcncaygarggnttyaaracnttyggnathwsnmgnaaymgnaaywsnmgnatgaayaargarccntgyttyttymgngcnatgytngtngtncayaarytnytnccnccngarytnytngcnatgtggggnytngtncaywsnaayytnacntgywsnmgnaarytncargtnytn

SEQ ID NO: 4 - protein, 6 aa, Artificial Sequence
  NSSP conserved peptide motif
XDVXXG

(19) United States
(12) Patent Application Publication
(10) Pub. No.: US 2003/0104398 A1
(43) Pub. Date: Jun. 5, 2003

(54) Galactosyltransferase homolog, ZNSSP11

(76) Inventors: David A. Adler, Bainbridge Island, WA (US); James L. Holloway, Seattle, WA (US); Gayle Yamamoto, Seattle, WA (US)

Correspondence Address: Robyn Adams, ZymoGenetics, Inc., 1201 Eastlake Avenue East, Seattle, WA, 98102, US

(21) Appl. No.: 10/015,725

(22) Filed: Nov. 2, 2001

Related U.S. Application Data
(60) Provisional application No. 60/245,516, filed on Nov. 3, 2000.

(51) Int. Cl. C12Q 1/68; C07H 21/04; C12N 9/10; C12P 21/02; C12N 5/06

(52) U.S. Cl. 435/6; 435/69.1; 435/193; 435/320.1; 435/325; 536/23.2

(57) ABSTRACT

Novel soluble, and membrane bound, Beta1,3 galactosyltransferase polypeptides, polynucleotides encoding the polypeptides, antibodies and related compositions and methods are disclosed. The polypeptides may be used for detecting anti-complementary molecules, agonists and antagonists. The polypeptides, polynucleotides and antibodies may also be used in methods that modulate cell-cell interactions, and glycoprotein and glycolipid modifications.

### REFERENCE TO RELATED APPLICATIONS

This application is related to Provisional Applications No. 60/245,516 filed on Nov. 3, 2000. Under 35 U.S.C. §119(e)(1), this application claims benefit of said Provisional Application.

### BACKGROUND OF THE INVENTION

Beta-1,3-galactosyltransferase molecules are classified in the family of glycosyltransferases. In addition to transferring carbohydrate molecules to glycoproteins during biosynthesis, members of this family have also been detected on the cell surface where they are thought to be involved in varying aspects of cell-cell interactions. This family includes carbohydrate transferring enzymes, such as sialyltransferases and fucosyltransferases, and galactosyltransferases. During the formation of O-linked glycoproteins and the modification of N-linked ones, each sugar transfer is catalyzed by a different type of glycosyltransferase. Each glycosyltransferase enzyme is specific for both the donor sugar nucleotide and the acceptor molecule.

Galactosyltransferases promote the transfer of an activated galactose residue in UDP-galactose to the monosaccharide N-acetylglucosamine. This transfer is a step in the biosynthesis of the carbohydrate portion of galactose-containing glycoproteins, such as oligosaccharides and glycolipids, in animal tissues. The Beta-1,3-galactosyl-transferases are characterized by the elongation of type I oligosaccharide chains, and the Beta-1,4-galactosyl-transferases are characterized by the elongation of type II oligosaccharide chains. Both types of carbohydrate structures are present in soluble oligosaccharides of human milk, and are also found on glycoproteins and glycolipids, and are important precursors of blood group antigens. Both galactosyltransferases require a divalent cation (Mn²⁺) to function. Beta-1,4-galactosyltransferases are expressed in various cell types and tissues, while the Beta-1,3-galactosyltransferases seem to have more restricted tissue distributions.

Some galactosyltransferases are found in the Golgi apparatus. These Golgi-localized enzymes have structure similarity: a short N-terminal domain that faces the cytosol, a single transmembrane α helix, and a large C-terminal domain that faces the Golgi lumen and that contains the catalytic site. The transmembrane α helix is necessary and sufficient to restrict the enzyme to the Golgi. Of the Beta-1,3-galactosyltransferase family two members (See Amado, M. et al., J. Biol. Chem. 273, 21: 12770-12778, 1998) have been predicted to have two potentially different initiation codons, resulting in two different N-terminal cytoplasmic domains.

Additionally, galactosyltransferases have been shown to be expressed on the cell surface, where their function is theorized to participate in cellular interactions, perhaps as receptors, or receptor-like complementary molecules. As a cell surface carbohydrate, galactosyltransferases have been implicated in varied biology such as cell migration, contact inhibition, tissue interactions, neuronal specificity, fertilization, embryonic cell adhesions, limb bud morphogenesis, mesenchyme development, immune recognition, growth control, and tumor metastasis. See, for example, Shur, B. D., Mol Cell Biol. 61:143-158, 1984.

The failure of tumor cell-tumor cell adhesion is believed to be a contributing factor to tumor metastases. See, for example, Zetter, Cancer Biology, 4: 219-29, 1993. Metastases, in turn, are generally associated with poor prognosis for cancer treatment. The metastatic process involves a variety of cellular events, including angiogenesis, tumor cell invasion of the vascular or lymphatic circulation, tumor cell arrest at a secondary site; tumor cell passage across the vessel wall into the parenchymal tissue, and tumor cell proliferation at the secondary site. Thus, both positive and negative regulation of adhesion are necessary for metastasis. That is, tumor cells must break away from the primary tumor mass, travel in circulation and adhere to cellular and/or extracellular matrix elements at a secondary site. Molecules capable of modulating cell-cell and cell-matrix adhesion are therefore sought for the study, diagnosis, prevention or treatment of metastases.

β1→3 Galactosyltransferases have limited homology to each other. In contrast to other glycosyltransferases, they do not appear to be localized to the same chromosomes. Additionally, a member of this family has recently been identified in Drosophila. This molecule, Brainiac, is involved in contact and adhesion between germ-line and follicle cells (Amado, M. et al., J. Biol. Chem. 273, 21: 12770-12778, 1998).

A deficiency of Beta-1,3-galactosyltransferase enzymes has been noticed in the Tn-syndrome. This syndrome is a rarely acquired disorder affecting all hemopoietic lineages, and is characterized by the expression of the Tn and the sialosyl-Tn antigens on the cell surface. The Tn is α N-acetylgalactosamine linked O-glycosidically to threonine or serine residues of membrane proteins. These antigens bind naturally occurring serum antibodies thereby leading to mild hemolytic anemia and pronounced thrombopenia. Thus, the blood cells in the Tn-syndrome are expected to carry less sialic acid if galactose can not be transferred to N-Acetylgalactosamine. The expression of Tn and sialosyl-Tn antigens as a consequence of imcomplete or disordered gylcan biosynthesis has been recognized as a cancer-associated phenomenon. Tn and sialosyl-Tn antigens are among the most investigated cancer-associated carbohydrates antigens.

The present invention provides such polypeptides for these and other uses that should be apparent to those skilled in the art from the teachings herein.

### DESCRIPTION OF THE INVENTION

Within one aspect the invention provides an isolated polypeptide comprising residues 151 to 415 of SEQ ID NO:2. Within an embodiment, the polypeptide comprises residues 42 to 415 of SEQ ID NO:2. Within another embodiment the polypeptide comprises residues 1 to 415 of SEQ ID NO:2. Within another embodiment, the invention provides an isolated polynucleotide encoding the polypeptide comprising residues 151 to 415 of SEQ ID NO:2. Within another embodiment, the polynucleotide molecule encodes a the polypeptide molecule chosen from: a polypeptide comprising residues 42 to 415 of SEQ ID NO:2; and a polypeptide comprising residues 1 to 415 of SEQ ID NO:2.

Within another aspect the invention provides an isolated polypeptide selected from the group consisting of: a polypeptide comprising residues 1 to 41 of SEQ ID NO:2; a polypeptide comprising residues 42 to 150 of SEQ ID NO:2; a polypeptide comprising residues 151 to 415 of SEQ ID NO:2; and a polypeptide comprising residues 42 to 415 of SEQ ID NO:2.

Within another aspect, is provided an expression vector comprising the following operably linked elements: a) a transcription promoter; b) a DNA segment wherein the DNA segment is a polynucleotide encoding the polypeptide comprising residues 151 to 415 of SEQ ID NO:2; and a transcription terminator. Within an embodiment, the the DNA segment contains an affinity tag. Within another embodiment is provides a cultured cell into which has been introduced the expression vector, wherein said cell expresses the polypeptide encoded by the DNA segment. Within another embodiment is provided a method of producing a polypeptide comprising culturing the cell, whereby said cell expresses the polypeptide encoded by the DNA segment; and recovering the polypeptide. Within another embodiment, the invention provides the polypeptide produced by the method.

Within another aspect antibodies to ZNSSP11 polypeptides are provided. Within one embodiment, is provided a method of producing an antibody comprising the following steps in order: inoculating an animal with a polypeptide selected from the group consisting of: a polypeptide comprising residues 1 to 41 of SEQ ID NO:2; a polypeptide comprising residues 42 to 150 of SEQ ID NO:2; a polypeptide comprising residues 151 to 415 of SEQ ID NO:2; a polypeptide comprising residues 42 to 415 of SEQ ID NO:2; and a polypeptide comprising residues 1 to 415 of SEQ ID NO:2, wherein the polypeptide elicits an immune response in the animal to produce the antibody; and isolating the antibody from the animal. Within another embodiment, the antibody produced by the method binds to a residues 1 to 415 of SEQ ID NO:2. Within another embodiment, the antibody is a monoclonal antibody. Within another embodiment, the antibody specifically binds to a polypeptide comprising the amino acid sequence as shown in SEQ ID NO:2.

Within another aspect the invention provides a method of producing an antibody comprising the following steps in order: inoculating an animal with an epitope bearing portion of a polypeptide wherein the epitope bearing portion is selected from the group consisting of: a polypeptide consisting of residues 49 to 69 of SEQ ID NO:2; a polypeptide consisting of residues 74 to 85 of SEQ ID NO:2; a polypeptide consisting of residues 88 to 95 of SEQ ID NO:2; a polypeptide consisting of residues 101 to 107 of SEQ ID NO:2; a polypeptide consisting of residues 141 148 of SEQ ID NO:2; a polypeptide consisting of residues 160 to 186 of SEQ ID NO:2; a polypeptide consisting of residues 194 to 203 of SEQ ID NO:2; a polypeptide consisting of residues 210 to 215 SEQ ID NO:2; a polypeptide consisting of residues 253 to 259 of SEQ ID NO:2; a polypeptide consisting of residues 269 to 275 of SEQ ID NO:2; a polypeptide consisting of residues 288 to 299 of SEQ ID NO:2; a polypeptide consisting of residues 304 314 of SEQ ID NO:2; a polypeptide consisting of residues 318 to 329 of SEQ ID NO:2; a polypeptide consisting of residues 355 to 362 of SEQ ID NO:2; a polypeptide consisting of residues 365 to 379 of SEQ ID NO:2; a polypeptide consisting of residues 405 to 413 of SEQ ID NO:2: a polypeptide consisting of residues 42 to 85 of SEQ ID NO:2; a polypeptide consisting of residues 52 to 66 of SEQ ID NO:2; a polypeptide consisting of residues 88 to 105 of SEQ ID NO:2; a polypeptide consisting of residues 109 to 118 of SEQ ID NO:2; a polypeptide consisting of residues 120 to 134 of SEQ ID NO:2; a polypeptide consisting of residues 139 to 147 of SEQ ID NO:2; a polypeptide consisting of residues 159 to 181 of SEQ ID NO:2; a polypeptide consisting of residues 195 to 217 of SEQ ID NO:2; a polypeptide consisting of residues 268 to 278 of SEQ ID NO:2;a polypeptide consisting of residues 284 to 299 of SEQ ID NO:2; a polypeptide consisting of residues 321 to 332 of SEQ ID NO:2; a polypeptide consisting of residues 355 to 362 of SEQ ID NO:2; a polypeptide consisting of residues 364 to 380 of SEQ ID NO:2: a polypeptide consisting of residues 50 to 67 of SEQ ID NO:2; a polypeptide consisting of residues 74 to 81 of SEQ ID NO:2; a polypeptide consisting of residues 161 to 168 of SEQ ID NO:2; a polypeptide consisting of residues 171 to 178 of SEQ ID NO:2; a polypeptide consisting of residues 195 to 205 of SEQ ID NO:2; a polypeptide consisting of residues 269 to 276 of SEQ ID NO:2;a polypeptide consisting of residues 286 to 298 of SEQ ID NO:2; and a polypeptide consisting of residues 367 to 376 of SEQ ID NO:2 wherein the polypeptide elicits an immune response in the animal to produce the antibody; and isolating the antibody from the animal. Within an embodiment, the antibody binds to a residues 1 to 415 of SEQ ID NO:2. Within another embodiment, the antibody is a monoclonal antibody.

Within another aspect of the invention, is provided, a method of forming a reversible complementary molecule-anti-complementary molecule complex comprising; providing an complementary molecule comprising residues 151 to 415 of SEQ ID NO:2; and contacting the complementary molecule with a anti-complementary molecule; whereby the complementary molecule binds the anti-complementary molecule. Within an embodiment, the anti-complementary molecule is selected from the group consisting of: glycoproteins; glycolipids; and antibodies.

Within another aspect the invention provides a method of detecting, diagnosing and treating disease and or tumorous tissues of colon, rectum, gallbladder, small intestine, and testis.

These and other aspects of the invention will become evident upon reference to the following detailed description of the invention.

Prior to setting forth the invention in detail, it may be helpful to the understanding thereof to define the following terms:

The term “affinity tag” is used herein to denote a polypeptide segment that can be attached to a second polypeptide to provide for purification of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985) (SEQ ID NO:7), substance P, Flag™ peptide (Hopp et al., Biotechnology 6:1204-1210, 1988), streptavidin binding peptide, maltose binding protein (Guan et al., Gene 67:21-30, 1987), cellulose binding protein, thioredoxin, ubiquitin, T7 polymerase, or other antigenic epitope or binding domain. See, in general, Ford et al., Protein Expression and Purification 2: 95-107, 1991. DNAs encoding affinity tags and other reagents are available from commercial suppliers (e.g., Pharmacia Biotech, Piscataway, N.J.; New England Biolabs, Beverly, Mass.; Eastman Kodak, New Haven, Conn.).

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complements of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

The term “corresponding to”, when applied to positions of amino acid residues in sequences, means corresponding positions in a plurality of sequences when the sequences are optimally aligned.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. The isolated polypeptide is substantially free of other polypeptides, particularly other polypeptides of animal origin. The polypeptides are provided in a highly purified form, i.e. greater than 95% pure, or greater than 99% pure. When used in this context, the term “isolated” does not exclude the presence of the same polypeptide in alternative physical forms, such as dimers or alternatively glycosylated or derivatized forms.

“Operably linked” means that two or more entities are joined together such that they function in concert for their intended purposes. When referring to DNA segments, the phrase indicates, for example, that coding sequences are joined in the correct reading frame, and transcription initiates in the promoter and proceeds through the coding segment(s) to the terminator. When referring to polypeptides, “operably linked” includes both covalently (e.g., by disulfide bonding) and non-covalently (e.g., by hydrogen bonding, hydrophobic interactions, or salt-bridge interactions) linked sequences, wherein the desired function(s) of the sequences are retained.

The term “ortholog” or “species homolog”, denotes a polypeptide or protein obtained from one species that is the functional counterpart of a polypeptide or protein from a different species. Sequence differences among orthologs are the result of speciation.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

A “soluble polypeptide” is a polypeptide that is not bound to a cell membrane. Soluble polypeptides lack a transmembrane and cytoplasmic domains. Soluble polypeptides can comprise additional amino acid residues, such as affinity tags that provide for purification of the polypeptide or provide sites for attachment of the polypeptide to a substrate. Many cell-surface polyeptides have naturally occurring, soluble counterparts that are produced by proteolysis or translated from alternatively spliced mRNAs. Enzyme polypeptides are said to be substantially free of transmembrane and intracellular polypeptide segments when they lack sufficient portions of these segments to provide membrane anchoring. For the purposes of this application a membrane-bound polypeptide may have intracellular capabilities.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based upon the discovery of a novel cDNA sequence (SEQ ID NO:1) and corresponding polypeptide (SEQ ID NO:2) having homology to a family of proteins, the β1→3galactosyltransferases (β1→3GalTases). β1→3GalTases are the β3 subfamily of human galactosyltransferases (β3Gal-T family) which includes HSY15014 (Kolbinger, F. et al., Journal of Biol. Chem. 273: 433-440, 1998), HSGALT3, HSGALT4, (Amado, M. et al., ibid) and E07739 (Katsutoshi, S. et al., Japanese patent, JP 1994181759-A/1). β1→3GalTases are responsible for transferring galactose to carbohydrate chains during biosynthesis. It has been predicted that β1→3GalTases are in the alpha/beta barrel (TIM barrel) folding class of enzymes, similar to other glycosyltransferases such as the alpha-amylases and beta-glycanases (Yuan, Y. et al., Cell 88:9-11, 1997). Also in the β3Gal-T family is the Drosophila melanogaster Brainiac, (BRN) (Goode, S. et al., Devel. Biol. 178:35-50, 1996), known as “putative neurogenic secreted signaling protein” or NSSP. BRN is required for epithelial development. This activity may be due to possible cell interactions between the membrane bound glycosyltransferase and oligosaccharide substrates on adjacent cell surfaces (Shur, ibid). Thus, β3Gal-T family members are also known as neurogenic secreted signal peptides. See, for example, Shur, B. D., ibid, and Amado, M. et al., ibid. This novel polypeptide and its polynucleotides have been designated ZNSSP11.

The β3Gal-Ts are predicted to be Type II transmembrane proteins. An ortholog to E07739, is AF029790 (Hennet, T. et al., Journal of Biol. Chem. 273:58-65, 1998), which is claimed to be a Type II transmembrane domain based on hydrophobicity analysis. However, due to the close proximity of this domain to the initiating methionine and lack of positively charged residues preceding the domain it is possible that AF029790 is not membrane bound but rather a extracellular secreted protein.

The novel ZNSSP11 polypeptide-encoding polynucleotides of the present invention were identified as a galactosyltrasnferase homolog. A full-length clone comprising nucleotide 1 to 1245 of SEQ ID NO:1 was identified in a cDNA library made from tissue containing an adenocarcinoma of colon and rectum. Analysis of the DNA encoding a ZNSSP11 polypeptide revealed an open reading frame encoding 415 amino acids (SEQ ID NO: 2). ZNSSP11 shares homology with β3Gal-T's. Amino acid residues 42 to 150 of SEQ ID NO:2 form a “stem” domain, and amino acid residues 151 to 415 of SEQ ID NO:2 form a “catalytic” domain.

Due to the close proximity of the hydrophobic domain (residues 1 to 41 of SEQ ID NO:2) to the initiation methionine, and the lack of positively charged residues preceding this domain, ZNSSP11 can be a secreted protein, or a membrane-bound protein. As a secreted protein, the mature polypeptide comprises residues 42 to 415 of SEQ ID NO:2. As a membrane bound protein, ZNSSP11 comprises residues 1 to 415 of SEQ ID NO:2. Conserved negatively charged amino acid residues 152, 219, 225, 234, 348, and 349 of SEQ ID NO:2 are contained within the catalytic domain. Additionally, the sequence of amino acid residues from residue 340 to 345 is representative of a peptide motif of this family. This motif is further described by the following amino acid residue profile: [D,E] [D] [V] [F,Y] [L,T,V,M] [G]. Those skilled in the art will recognize that predicted domain boundaries are approximations based on primary sequence content, and may vary slightly; however, such estimates are generally accurate to within \mathring{6}5 amino acid residues.

The present invention also provides post translationally modified polypeptides or polypeptide fragments. Post translational modifications in members of the β3Gal-T family may regulate whether the protein is expressed in the Golgi or on the surface of the cell. Other examples of post translational modifications include proteolytic cleavage, disulfide bonding and hydroxylation.

The highly conserved, negatively charged residues at positions 152, 219, 225, 234, 348, and 349 of SEQ ID NO:2 and the amino acid sequence between 340 and 345 of ZNSSP11 can be used as a tool to identify new family members. For instance, reverse transcription-polymerase chain reaction (RT-PCR) can be used to amplify sequences encoding the ZNSSP11 polynucleotide from RNA obtained from a variety of tissue sources or cell lines. In particular, highly degenerate primers designed from the ZNSSP11 sequences are useful for this purpose.

Analysis of the tissue distribution of ZNSSP11 can be performed by the Northern blotting technique using Human Multiple Tissue and Master Dot Blots. Such blots are commercially available (Clontech, Palo Alto, Calif.) and can be probed by methods known to one skilled in the art. Also see, for example, Wu W. et al., Methods in Gene Biotechnology, CRC Press LLC, 1997. Additionally, querying sequence databases and identifying the tissues from which the sequences are derived can identify portions of the polynucleotides of the present invention. Portions of the polynucleotides of the present invention have been identified in cDNA libraries that have been prepared from diseased and tumorous tissues from colon/rectum (adenocarcinoma), small intestine (chronic ulcerative colitis), gallbladder (chronic cholecystitis), and testis (embryonal carcinoma). Additionally, portions of the polynucleotide sequence have been identified in cDNA libraries prepared from a human teratocarcinoma cell line. Portions of the polynucleotide sequence have also been identified in cDNA libraries prepared from normal brain, mixed (myometrium and smooth muscle) tissues, and normal colon.

The failure of tumor cell-tumor cell adhesion is believed to be a contributing factor in tumor metastases. See, for example, Zetter, Cancer Biology, 4: 219-29, 1993. Metastases, in turn, are generally associated with poor prognosis for cancer treatment. The metastatic process involves a variety of cellular events, including angiogenesis, tumor cell invasion of the vascular or lymphatic circulation, tumor cell arrest at a secondary site; tumor cell passage across the vessel wall into the parenchymal tissue, and tumor cell proliferation at the secondary site. Thus, both positive and negative regulation of adhesion are necessary for metastasis. That is, tumor cells must break away from the primary tumor mass, travel in circulation and adhere to cellular and/or extracellular matrix elements at a secondary site. Molecules capable of modulating cell-cell and cell-matrix adhesion are therefore sought for the study, diagnosis, prevention and/or treatment of metastases.

Beta-1,3-galactosyltransferases have limited homology to each other. In contrast to other glycosyltransferases, they do not appear to be localized to the same chromosomes. Additionally, a member of this family has recently been identified in Drosophila. This molecule, Brainiac (brn), also known as a Neurogenic Secreted Signaling Peptide (NSSP), is involved in contact and adhesion between germ-line and follicle cells (Amado, M. et al., J. Biol. Chem. 273, 21: 12770-12778, 1998). Germline Brainiac activity has been shown to be essential for development of follicular epithelium (Goode, S. et al., Dev. Biol. 178:35-50, 1996). Additionally, brn is required continuously throughout oogenesis, beginning in the germarium at the time that follicle cells envelop the oocyte-nurse cell complex and continuing stages when the eggshell is produced. The expression of brn in the germline continuously throughout oogenesis is consistent with brn's role in developing the follicular epithelium around each germline cyst, as well as for dorsal-ventral patterning of the follicular epithelium during later phases of oogenesis. See Goode, S. et al., Development. 116: 177-192, 1992.

The present invention further provides polynucleotide molecules, including DNA and RNA molecules, encoding ZNSSP11 proteins. The polynucleotides of the present invention include the sense strand; the anti-sense strand; and the DNA as double-stranded, having both the sense and anti-sense strand annealed together by their respective hydrogen bonds. Representative DNA sequences encoding ZNSSP11 proteins are set forth in SEQ ID NOs:1 and 3. DNA sequences encoding other ZNSSP11 proteins can be readily generated by those of ordinary skill in the art based on the genetic code.

The present invention provides polynucleotide molecules, including DNA and RNA molecules, that encode the ZNSSP11 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the ZNSSP11 polypeptide of SEQ ID NO:2. Those skilled in the art will recognize that the degenerate sequence of SEQ ID NO:3 also provides all RNA sequences encoding SEQ ID NO:2 by substituting U for T. Thus, ZNSSP11 polypeptide-encoding polynucleotides comprising nucleotide 1 to nucleotide 2003 of SEQ ID NO:1, and their RNA equivalents are contemplated by the present invention. Table 1 sets forth the one-letter codes used within SEQ ID NO:3 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Nucleotide | Complement |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:3, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed in SEQ ID NO:3 serve as templates for optimizing expression of polynucleotides in various cell types and species commonly used in the art and disclosed herein. Sequences containing preferential codons can be tested and optimized for expression in various species, and tested for functionality as disclosed herein.

Within embodiments of the invention the isolated polynucleotides will hybridize to similar sized regions of SEQ ID NO:1, or a sequence complementary thereto under stringent conditions. Polynucleotide hybridization is well known in the art and widely used for many applications, see for example, Sambrook et al., Molecular Cloning: A Laboratory Manual, Second Edition, Cold Spring Harbor, N.Y., 1989;Ausubel et al., eds., Current Protocols in Molecular Biology, John Wiley and Sons, Inc., NY, 1987; Berger and Kimmel, eds., Guide to Molecular Cloning Techniques, Methods in Enzymology, volume 152, 1987 and Wetmur, Crit. Rev. Biochem. Mol. Biol. 26:227-59, 1990. Polynucleotide hybridization exploits the ability of single stranded complementary sequences to form a double helix hybrid. Such hybrids include DNA-DNA, RNA-RNA and DNA-RNA.

As an illustration, a nucleic acid molecule encoding a variant ZNSSP11 polypeptide can be hybridized with a nucleic acid molecule having the nucleotide sequence of SEQ ID NO:1 (or its complement) at 65° C. overnight in ExpressHyb™ Hybridization Solution (CLONTECH Laboratories, Inc., Palo Alto, Calif.). One of skill in the art can devise variations of these hybridization conditions.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding a variant ZNSSP11 polypeptide hybridize with a nucleic acid molecule having the nucleotide sequences of SEQ ID NO:1 (or its complement) under stringent washing conditions, in which the wash stringency is equivalent to 0.1×-2×SSC with 0.1% SDS at 55-65° C., including 0.1×SSC with 0.1% SDS at 55° C., or 2×SSC with 0.1% SDS at 65° C. One of skill in the art can readily devise equivalent conditions, for example, by substituting SSPE for SSC in the wash solution.

The present invention also contemplates ZNSSP11 variant nucleic acid molecules that can be identified using two criteria: a determination of the similarity between the encoded polypeptides with the amino acid sequences of SEQ ID NO:2 (as described below), and a hybridization assay, as described above. Such ZNSSP11 variants include nucleic acid molecules that hybridize with a nucleic acid molecule having the nucleotide sequence of SEQ ID NO:1 (or its complement) under stringent washing conditions, in which the wash stringency is equivalent to 0.1×-2×SSC with 0.1% SDS at 55-65° C., and (2) that encode a polypeptide having about 80%, 90%, 95% or greater than 95% sequence identity to the amino acid sequence of SEQ ID NO:2. Alternatively, ZNSSP11 variants can be characterized as nucleic acid molecules (1) that hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C., and (2) that encode a polypeptide having about 80%, 90%, 95% or greater than 95% sequence identity to the amino acid sequence of SEQ ID NO:2.

The highly conserved amino acids in the catalytic domain of ZNSSP11 can be used as a tool to identify new family members. For instance, reverse transcription-polymerase chain reaction (RT-PCR) can be used to amplify sequences encoding the conserved catalytic domain from RNA obtained from a variety of tissue sources or cell lines. In particular, highly degenerate primers designed from the ZNSSP11 sequences are useful for this purpose.

As previously noted, the isolated polynucleotides of the present invention include DNA and RNA. Methods for preparing DNA and RNA are well known in the art. In general, RNA is isolated from a tissue or cell that produces ZNSSP11 RNA. Such tissues and cells can be identified by Northern blotting (Thomas, Proc. Natl. Acad. Sci. USA 77:5201, 1980), and include tissues of diseased and/or tumorous colon, rectum, gallbladder, small intestine, and testis, as well as normal colon and brain.

Total RNA can be prepared using guanidine isothiocyante extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding ZNSSP11 polypeptides are then identified and isolated by, for example, hybridization or PCR.

A full-length clone encoding ZNSSP11 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to ZNSSP11 or other specific binding partners.

The invention also provides isolated and purified ZNSSP11 polynucleotide probes. Such polynucleotide probes can be RNA or DNA. DNA can be either cDNA or genomic DNA. Polynucleotide probes are single or double-stranded DNA or RNA, generally synthetic oligonucleotides, but may be generated from cloned cDNA or genomic sequences and will generally comprise at least 16 nucleotides, more often from 17 nucleotides to 25 or more nucleotides, sometimes 40 to 60 nucleotides, and in some instances a substantial portion, domain or even the entire ZNSSP11 gene or cDNA. The synthetic oligonucleotides of the present invention have at least 75% identity to a representative ZNSSP11 DNA sequence (SEQ ID NOS:1 or 3) or their complements. The invention also provides oligonucleotide probes or primers comprising at least 14 contiguous nucleotides of a polynucleotide of SEQ ID NOs: 1 or 3 or a sequence complementary to SEQ ID NOs: 1 or 3.

Regions from which to construct probes include the 5′ and/or 3′ coding sequences, anti-complementary molecule binding regions, and signal sequences, and the like. Techniques for developing polynucleotide probes and hybridization techniques are known in the art, see for example, Ausubel et al., eds., Current Protocols in Molecular Biology, John Wiley and Sons, Inc., NY, 1991. For use as probes, the molecules can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art. Such probes can also be used in hybridizations to detect the presence or quantify the amount of ZNSSP11 gene or mRNA transcript in a sample. ZNSSP11 polynucleotide probes could be used to hybridize to DNA or RNA targets for diagnostic purposes, using such techniques such as fluorescent in situ hybridization (FISH) or immunohistochemistry. Polynucleotide probes can be used to identify genes encoding ZNSSP11-like proteins. For example, ZNSSP11 polynucleotides can be used as primers and/or templates in PCR reactions to identify other novel members of the Beta1,3 galactosyltransferase family. Such probes can also be used to screen libraries for related sequences encoding novel Beta1,3 galactosyltransferases. Such screening would be carried out under conditions of low stringency which would allow identification of sequences which are substantially homologous, but not requiring complete homology to the probe sequence. Such methods and conditions are well known in the art, see, for example, Sambrook et al., Molecular Cloning: A Laboratory Manual, Second Edition, Cold Spring Harbor, N.Y., 1989. Such low stringency conditions could include hybridization temperatures less than 42° C., formamide concentrations of less than 50% and moderate to low concentrations of salt. Libraries may be made of genomic DNA or cDNA. Polynucleotide probes are also useful for Southern, Northern, or dot blots, colony and plaque hybridization and in situ hybridization. Mixtures of different ZNSSP11 polynucleotide probes can be prepared which would increase sensitivity or the detection of low copy number targets, in screening systems.

In addition, such polynucleotide probes could be used to hybridize to counterpart sequences on individual chromosomes. Chromosomal identification and/or mapping of the ZNSSP11 gene could provide useful information about gene function and disease association. Many mapping techniques are available to one skilled in the art, for example, mapping somatic cell hybrids, and fluorescence in situ hybridization (FISH). One method is radiation hybrid mapping. Radiation hybrid mapping is a somatic cell genetic technique developed for constructing high-resolution, contiguous maps of mammalian chromosomes (Cox et al., Science 250:245-50, 1990). Partial or full knowledge of a gene's sequence allows the designing of PCR primers suitable for use with chromosomal radiation hybrid mapping panels. Commercially available radiation hybrid mapping panels which cover the entire human genome, such as the Stanford G3 RH Panel and the GeneBridge 4 RH Panel (Research Genetics, Inc., Huntsville, Ala.), are available. These panels enable rapid, PCR based, chromosomal localizations and ordering of genes, sequence-tagged sites (STSs), and other non-polymorphic- and polymorphic markers within a region of interest. This includes establishing directly proportional physical distances between newly discovered genes of interest and previously mapped markers. The precise knowledge of a gene's position can be useful in a number of ways including: 1) determining if a sequence is part of an existing contig and obtaining additional surrounding genetic sequences in various forms such as YAC-, BAC- or cDNA clones, 2) providing a possible candidate gene for an inheritable disease which shows linkage to the same chromosomal region, and 3) for cross-referencing model organisms such as mouse which may be beneficial in helping to determine what function a particular gene might have.

ZNSSP11 polynucleotide sequences disclosed herein can also be used as probes or primers to clone 5′ non-coding regions of a ZNSSP11 gene. In view of the tissue-specific expression observed for ZNSSP11 by Northern blotting, this gene region is expected to provide for specific expression in tissues of the include tissues of diseased and/or tumorous colon, rectum, gallbladder, small intestine, and testis, as well as normal colon and brain. Promoter elements from a ZNSSP11 gene could thus be used to direct the tissue-specific expression of heterologous genes in, for example, transgenic animals or patients treated with gene therapy. Cloning of 5′ flanking sequences also facilitates production of ZNSSP11 proteins by “gene activation” as disclosed in U.S. Pat. No. 5,641,670. Briefly, expression of an endogenous ZNSSP11 gene in a cell is altered by introducing into the ZNSSP11 locus a DNA construct comprising at least a targeting sequence, a regulatory sequence, an exon, and an unpaired splice donor site. The targeting sequence is a ZNSSP11 5′ non-coding sequence that permits homologous recombination of the construct with the endogenous ZNSSP11 locus, whereby the sequences within the construct become operably linked with the endogenous ZNSSP11 coding sequence. In this way, an endogenous ZNSSP11 promoter can be replaced or supplemented with other regulatory sequences to provide enhanced, tissue-specific, or otherwise regulated expression.

The polynucleotides of the present invention can also be synthesized using DNA synthesizers. Currently the method of choice is the phosphoramidite method. If chemically synthesized double stranded DNA is required for an application such as the synthesis of a gene or a gene fragment, then each complementary strand is made separately. The production of short genes (60 to 80 bp) is technically straightforward and can be accomplished by synthesizing the complementary strands and then annealing them. For the production of longer genes (>300 bp), however, special strategies must be invoked, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. See Glick and Pasternak, Molecular Biotechnology, Principles and Applications of Recombinant DNA, (ASM Press, Washington, D.C. 1994); Itakura et at., Annu. Rev. Biochem. 53: 323-356 (1984) and Climie et al., Proc. Natl. Acad. Sci. USA 87:633-7, 1990.

The present invention further provides counterpart polypeptides and polynucleotides from other species (orthologs). These species include, but are not limited to mammalian, avian, amphibian, reptile, fish, insect and other vertebrate and invertebrate species. Of particular interest are ZNSSP11 polypeptides from other mammalian species, including murine, porcine, ovine, bovine, canine, feline, equine, and other primate polypeptides. Orthologs of human ZNSSP11 can be cloned using information and compositions provided by the present invention in combination with conventional cloning techniques. For example, a cDNA can be cloned using mRNA obtained from a tissue or cell type that expresses ZNSSP11 as disclosed herein. Such tissues and cell types would include, for example, tissues of diseased and/or tumorous colon, rectum, gallbladder, small intestine, and testis, as well as normal colon and brain. Suitable sources of mRNA can be identified by probing Northern blots with probes designed from the sequences disclosed herein. A library is then prepared from mRNA of a positive tissue or cell line. A ZNSSP11-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human ZNSSP11 sequences disclosed herein. Within an additional method, the cDNA library can be used to transform or transfect host cells, and expression of the cDNA of interest can be detected with an antibody to ZNSSP11 polypeptide. Similar techniques can also be applied to the isolation of genomic clones.

Those skilled in the art will recognize that the sequences disclosed in SEQ ID NO:1 represents a single allele of human ZNSSP11 and that allelic variation and alternative splicing are expected to occur. Allelic variants of this sequence can be cloned by probing cDNA or genomic libraries from different individuals according to standard procedures. Allelic variants of the DNA sequences shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the ZNSSP11 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art. As stated earlier, polynucleotides of SEQ ID NO:1 and SEQ ID NO:3 are alternatively spliced variants of the same gene.

The present invention also provides isolated ZNSSP11 polypeptides that are substantially similar to the polypeptides of SEQ ID NO:2 and their orthologs. Such polypeptides will be about 80%, 90% identical, or 95% or more identical to SEQ ID NO:2 and their orthologs. Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant ZNSSP11. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from four to six.

The present invention includes nucleic acid molecules that encode a polypeptide having one or more conservative amino acid changes, compared with the amino acid sequences of SEQ ID NO:2. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. As used herein, the language “conservative amino acid substitution” refers to a substitution represented by a BLOSUM62 value of greater than −1. For example, an amino acid substitution is conservative if the substitution is characterized by a BLOSUM62 value of 0, 1, 2, or 3. Conservative amino acid substitutions are characterized by a BLOSUM62 value of at least 1 (e.g., 1, 2 or 3), while more conservative amino acid substitutions are characterized by a BLOSUM62 value of at least 2 (e.g., 2 or 3).

Conservative amino acid changes in an ZNSSP11 gene can be introduced by substituting nucleotides for the nucleotides recited in SEQ ID NO:1. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)). The ability of such variants to promote cell-cell interactions can be determined using a standard method, such as the assay described herein. Alternatively, a variant ZNSSP11 polypeptide can be identified by the ability to specifically bind anti-ZNSSP11 antibodies.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of complementary molecule-anti-complementary molecule interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related Beta1,3 galactosyltransferase-like molecules.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed ZNSSP11 DNA and polypeptide sequences can be generated through DNA shuffling, as disclosed by Stemmer, Nature 343:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., anti-complementary molecule binding activity) can be recovered from the host cells and rapidly sequenced using modern equipment. These methods allow the rapid determination of the importance of individual amino acid residues in a polypeptide of interest, and can be applied to polypeptides of unknown structure.

Regardless of the particular nucleotide sequence of a variant ZNSSP11 gene, the gene encodes a polypeptide that is characterized by its anti-complementary molecule binding activity, or by the ability to bind specifically to an anti-ZNSSP11 antibody. More specifically, variant ZNSSP11 genes encode polypeptides which exhibit greater than 75, 80, or 90%, of the activity of polypeptide encoded by the human ZNSSP11 gene described herein.

Variant ZNSSP11 polypeptides or substantially homologous ZNSSP11 polypeptides are characterized as having one or more amino acid substitutions, deletions or additions. These changes are of a minor nature, that is conservative amino acid substitutions and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from 288 to 1650 amino acid residues that comprise a sequence that is about 85%, 90%, and 95% or more identical to the corresponding region of SEQ ID NO:2. Polypeptides comprising affinity tags can further comprise a proteolytic cleavage site between the ZNSSP11 polypeptide and the affinity tag. Such sites include thrombin cleavage sites and factor Xa cleavage sites.

For any ZNSSP11 polypeptide, including variants and fusion proteins, one of ordinary skill in the art can readily generate a fully degenerate polynucleotide sequence encoding that variant using the information set forth in Tables 1 and 2 above. Moreover, those of skill in the art can use standard software to devise ZNSSP11 variants based upon the nucleotide and amino acid sequences described herein. Accordingly, the present invention includes a computer-readable medium encoded with a data structure that provides at least one of the following sequences: SEQ ID NO: 1, SEQ ID NO:2, and SEQ ID NO:3. Suitable forms of computer-readable media include magnetic media and optically-readable media. Examples of magnetic media include a hard or fixed drive, a random access memory (RAM) chip, a floppy disk, digital linear tape (DLT), a disk cache, and a ZIP disk. Optically readable media are exemplified by compact discs (e.g., CD-read only memory (ROM), CD-rewritable (RW), and CD-recordable), and digital versatile/video discs (DVD) (e.g., DVD-ROM, DVD-RAM, and DVD+RW).

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. For example, part or all of a domain(s) conferring a biological function may be swapped between ZNSSP11 of the present invention with the functionally equivalent domain(s) from another family member, such as HSGALT3, HSGALT4, β3Gal-T2, and β3Gal-T3, or the human species ortholog of Brainiac. Such domains include, but are not limited to, conserved motifs such as the signal, transmembrane, stem, and catalytic domains. Such fusion proteins would be expected to have a biological functional profile that is the same or similar to polypeptides of the present invention or other known Beta-1,3-galactosyltransferase family proteins (e.g. HSGALT3, HSGALT4, β3Gal-T2, and β3Gal-T3, or the human species ortholog of Brainiac), depending on the fusion constructed. Moreover, such fusion proteins may exhibit other properties as disclosed herein.

Moreover, using methods described in the art, polypeptide fusions, or hybrid ZNSSP11 proteins, are constructed using regions or domains of the inventive ZNSSP11 in combination with those of other Beta-1,3-galactosyltransferase molecules (e.g. HSGALT3, HSGALT4, β3Gal-T2, and β3Gal-T3, or the human species ortholog of Brainiac), or heterologous proteins (Sambrook et al., ibid., Altschul et al., ibid., Picard, Cur. Opin. Biology, 5:511-5, 1994, and references therein). These methods allow the determination of the biological importance of larger domains or regions in a polypeptide of interest. Such hybrids may alter reaction kinetics, binding, constrict or expand the anti-complementary molecule specificity, or alter tissue and cellular localization of a polypeptide, and can be applied to polypeptides of unknown structure.

Auxiliary domains can be fused to ZNSSP11 polypeptides to target them to specific cells, tissues, or macromolecules (e.g., tissues of diseased and/or tumorous colon, rectum, gallbladder, small intestine, and testis, as well as normal colon and brain). For example, a protease domain could be targeted to a predetermined cell type (tissues of diseased and/or tumorous colon, rectum, gallbladder, small intestine, and testis, as well as normal colon and brain) by fusing it to the catalytic domain (residues 151 to 403 of SEQ ID NO:2), or a portion thereof. In this way, polypeptides, polypeptide fragments and proteins can be targeted for therapeutic or diagnostic purposes. Such catalytic domain, or portions thereof can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

Polypeptide fusions of the present invention will generally contain not more than about 1,700 amino acid residues, not more than about 1,200 residues, or not more than about 1,000 residues, and will in many cases be considerably smaller. For example, residues of ZNSSP11 polypeptide can be fused to E. coli β-galactosidase (1,021 residues; see Casadaban et al., J. Bacteriol. 143:971-980, 1980), a 10-residue spacer, and a 4-residue factor Xa cleavage site. In a second example, residues of ZNSSP11 polypeptide can be fused to maltose binding protein (approximately 343 residues), a 4-residue cleavage site, and a 6-residue polyhistidine tag.

Some proteins in the β3Gal-T family been shown to be expressed intracellulary and are involved in intracellular glycoprotein and glycolipid processing. Other members of this family have been shown to be extracellularly expressed and are involved in glycoprotein and glycolipid processing (such as in the case of the Tn antigen). Other members of the family are expressed extracellularly and are involved in cell-cell interactions and intracellular signaling. Thus, molecules of the present invention can function as an enzyme both intracellularly and extracellulary, in which case its anti-complementary molecule is a substrate. Additionally, molecules of the present invention can function extracellularly and modulate cell-cell interactions. The extracellular binding of ZNSSP11 to its anti-complementary molecule can cause a cellular event in the cell that is expressing it (i.e. ZNSSP11 acts as a receptor or receptor-like molecule), or in the cell expressing the anti-complementary molecule to which it binds (i.e., ZNSSP11 acts as a ligand). Additionally, ZNSSP11 can function extracellularly as a soluble enzyme, ligand, receptor or receptor like molecule. Similarly, as an extracellulary expressed ZNSSP11 enzyme, the processing of its anti-complementary substrate can result in a cellular response (similar to intracellular signaling) in the cell expressing the substrate. Also as an extracellularly expressed protein, ZNSSP11 can function to form a “bridge” between cells maintaining their proximity to each other. Thus, for the purposes of this application, ZNSSP11 is referred to as a complementary molecule and its cognate binding partner is referred to as an anti-complementary molecule.

The invention also provides soluble ZNSSP11 polypeptides, used to form fusion or chimeric proteins with human Ig, as His-tagged proteins, or FLAG™-tagged proteins. One such construct is comprises residues 151 to 403 of SEQ ID NO:2, fused to human Ig. ZNSSP11 or ZNSSP11-Ig chimeric proteins are used, for example, to identify the ZNSSP11 anti-complementary molecules, including the natural anti-complementary molecule, as well as agonists and antagonists of the natural anti-complementary molecule. Using labeled soluble ZNSSP11, cells expressing the anti-complementary molecule are identified by fluorescence immunocytometry or immunohistochemistry. The soluble fusion proteins or soluble Ig fusion protein is useful in studying the distribution of the anti-complementary molecule on tissues or specific cell lineages, and to provide insight into complementary molecule-anti-complementary molecule biology.

In an alternative approach, a soluble ZNSSP11 extracellular anti-complementary molecule-binding region can be expressed as a chimera with immunoglobulin heavy chain constant regions, typically an Fc fragment, which contains two constant region domains and a hinge region, but lacks the variable region. Such fusions are typically secreted as multimeric molecules, wherein the Fc portions are disulfide bonded to each other and two complementary molecule polypeptides are arrayed in close proximity to each other. Fusions of this type can be used to affinity purify the cognate anti-complementary molecule from solution, as an in vitro assay tool, to block signals in vitro by specifically titrating out anti-complementary molecule, and as antagonists in vivo by administering them to block anti-complementary molecule stimulation. To purify anti-complementary molecule, a ZNSSP11-Ig fusion protein (chimera) is added to a sample containing the anti-complementary molecule under conditions that facilitate complementary molecule-anti-complementary molecule binding (typically near-physiological temperature, pH, and ionic strength). The chimera-anti-complementary molecule complex is then separated by the mixture using protein A, which is immobilized on a solid support (e.g., insoluble resin beads). The anti-complementary molecule is then eluted using conventional chemical techniques, such as with a salt or pH gradient. In the alternative, the chimera itself can be bound to a solid support, with binding and elution carried out as above. For use in assays, the chimeras are bound to a support via the F_{c} region and used in an ELISA format.

To direct the export of a ZNSSP11 polypeptide from the host cell, the ZNSSP11 DNA is linked to a second DNA segment encoding a secretory peptide, such as a t-PA secretory peptide. To facilitate purification of the secreted polypeptide, a C-terminal extension, such as a poly-histidine tag, substance P, Flag peptide (Hopp et al., Bio/Technology 6:1204-1210, 1988; available from Eastman Kodak Co., New Haven, Conn.), maltose binding protein, or another polypeptide or protein for which an antibody or other specific binding agent is available, can be fused to the ZNSSP11 polypeptide.

The present invention also includes “functional fragments” of ZNSSP11 polypeptides and nucleic acid molecules encoding such functional fragments. Routine deletion analyses of nucleic acid molecules can be performed to obtain functional fragments of a nucleic acid molecule that encodes an ZNSSP11 polypeptide. As an illustration, DNA molecules having the nucleotide sequence of SEQ ID NO:1 can be digested with Bal31 nuclease to obtain a series of nested deletions. The fragments are then inserted into expression vectors in proper reading frame, and the expressed polypeptides are isolated and tested for cell-cell interactions, or for the ability to bind anti-ZNSSP11 antibodies. One alternative to exonuclease digestion is to use oligonucleotide-directed mutagenesis to introduce deletions or stop codons to specify production of a desired fragment. Alternatively, particular fragments of an ZNSSP11 gene can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993), Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987), Herschman, “The EGF Enzyme,” in Control of Animal Cell Proliferation, Vol. 1, Boynton et al., (eds.) pages 169-199 (Academic Press 1985), Coumailleau et al., J. Biol. Chem. 270:29270 (1995); Fukunaga et al., J. Biol. Chem. 270:25291 (1995); Yamaguchi et al., Biochem. Pharmacol. 50:1295 (1995), and Meisel et al., Plant Molec. Biol. 30:1 (1996).

The present invention also contemplates functional fragments of an ZNSSP11 gene that has amino acid changes, compared with the amino acid sequence of SEQ ID NO:2. A variant ZNSSP11 gene can be identified on the basis of structure by determining the level of identity with nucleotide and amino acid sequences of SEQ ID NOs:1 and 2, as discussed above. An alternative approach to identifying a variant gene on the basis of structure is to determine whether a nucleic acid molecule encoding a potential variant ZNSSP11 gene can hybridize to a nucleic acid molecule having the nucleotide sequence of SEQ ID NO:1, as discussed above.

Using the methods discussed herein, one of ordinary skill in the art can identify and/or prepare a variety of polypeptide fragments or variants of SEQ ID NO:2 or that retain the glycosyltransferase and/or the cell-cell interaction activity of the wild-type ZNSSP11 protein. Such polypeptides may include additional amino acids from, for example, a secretory signal, transmembrane, stem, and catalytic domains, including amino acids responsible for intracellular signaling; fusion domains; affinity tags; and the like.

Within the polypeptides of the present invention are polypeptides that comprise an epitope-bearing portion of a protein as shown in SEQ ID NO:2. An “epitope” is a region of a protein to which an antibody can bind. See, for example, Geysen et al., Proc. Natl. Acad. Sci. USA 81:3998-4002, 1984. Epitopes can be linear or conformational, the latter being composed of discontinuous regions of the protein that form an epitope upon folding of the protein. Linear epitopes are generally at least 6 amino acid residues in length. Relatively short synthetic peptides that mimic part of a protein sequence are routinely capable of eliciting an antiserum that reacts with the partially mimicked protein. See, Sutcliffe et al., Science 219:660-666, 1983. Antibodies that recognize short, linear epitopes are particularly useful in analytic and diagnostic applications that employ denatured protein, such as Western blotting (Tobin, Proc. Natl. Acad. Sci. USA 76:4350-4356, 1979), or in the analysis of fixed cells or tissue samples. Antibodies to linear epitopes are also useful for detecting fragments of ZNSSP11, such as might occur in body fluids or cell culture media.

Antigenic, epitope-bearing polypeptides of the present invention are useful for raising antibodies, including monoclonal antibodies, that specifically bind to a ZNSSP11 protein. Antigenic, epitope-bearing polypeptides contain a sequence of at least six, or at least nine, or from 15 to about 30 contiguous amino acid residues of a ZNSSP11 protein (e.g., SEQ ID NO:2). Polypeptides comprising a larger portion of a ZNSSP11 protein, i.e. from 30 to 50 residues up to the entire sequence, are included. The amino acid sequence of the epitope-bearing polypeptide can be selected to provide substantial solubility in aqueous solvents that is the sequence includes relatively hydrophilic residues, and hydrophobic residues are substantially avoided. Such regions include the signal, transmembrane, stem, and catalytic domains of ZNSSP11 and fragments thereof. Polypeptides in this regard include those comprising residues 1 to 42 of SEQ ID NO:2; residues 47 to 150 of SEQ ID NO:2; residues 151 to 403 of SEQ ID NO:2; residues 151 to 415 of SEQ ID NO:2; residues 43 to 403 of SEQ ID NO:2; residues 43 to 415 of SEQ ID NO:2; residues 1 to 403 of SEQ ID NO:2; residues 1 to 415 of SEQ ID NO:2; and residues 1 to 150 of SEQ ID NO:2.

The present invention also provides polypeptide fragments or peptides comprising an epitope-bearing portion of an ZNSSP11 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein.

Antigenic epitope-bearing peptides and polypeptides contain at least four to ten amino acids, or at least ten to fifteen amino acids, or 15 to 30 amino acids of SEQ ID NO:2. Such epitope-bearing peptides and polypeptides can be produced by fragmenting an ZNSSP11 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

ZNSSP11 polypeptides can also be used to prepare antibodies that specifically bind to ZNSSP11 epitopes, peptides or polypeptides. The ZNSSP11 polypeptide or a fragment thereof serves as an antigen (immunogen) to inoculate an animal and elicit an immune response. One of skill in the art would recognize that antigenic, epitope-bearing polypeptides contain a sequence of at least 6, or at least 9, and at least 15 to about 30 contiguous amino acid residues of a ZNSSP11 polypeptide (e.g., SEQ ID NO:2). Polypeptides comprising a larger portion of a ZNSSP11 polypeptide, i.e., from 30 to 10 residues up to the entire length of the amino acid sequence are included. Antigens or immunogenic epitopes can also include attached tags, adjuvants and carriers, as described herein. Suitable antigens include the ZNSSP11 polypeptides encoded by SEQ ID NO:2 from amino acid number 1 to amino acid number 415, or a contiguous 9 to 415 amino acid fragment thereof.

As an illustration, potential antigenic sites in ZNSSP11 were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

Suitable antigens include residue 49 to residue 69 of SEQ ID NO:2; residue 74 to residue 85 of SEQ ID NO:2; residue 88 to residue 95 of SEQ ID NO:2; residue 101 to residue 107 of SEQ ID NO:2; residue 141 residue 148 of SEQ ID NO:2; residue 160 to residue 186 of SEQ ID NO:2; residue 194 to residue 203 of SEQ ID NO:2; residue 210 to residue 215 SEQ ID NO:2; residue 253 to residue 259 of SEQ ID NO:2; residue 269 to residue 275 of SEQ ID NO:2; residue 288 to residue 299 of SEQ ID NO:2; residue 304 residue 314 of SEQ ID NO:2; residue 318 to residue 329 of SEQ ID NO:2; residue 355 to residue 362 of SEQ ID NO:2; residue 365 to residue 379 of SEQ ID NO:2; and residue 405 to residue 413 of SEQ ID NO:2.. Hydrophilic peptides, such as those predicted by one of skill in the art from a hydrophobicity plot are also immonogenic. ZNSSP11 hydrophilic peptides include peptides comprising amino acid sequences selected from the group consisting of: residue 42 to residue 85 of SEQ ID NO:2; residue 52 to residue 66 of SEQ ID NO:2; residue 88 to residue 105 of SEQ ID NO:2; residue 109 to residue 118 of SEQ ID NO:2; residue 120 to residue 134 of SEQ ID NO:2; residue 139 to residue 147 of SEQ ID NO:2; residue 159 to residue 181 of SEQ ID NO:2; residue 195 to residue 217 of SEQ ID NO:2; residue 268 to residue 278 of SEQ ID NO:2; residue 284 to residue 299 of SEQ ID NO:2; residue 321 to residue 332 of SEQ ID NO:2; residue 355 to residue 362 of SEQ ID NO:2; and residue 364 to residue 380 of SEQ ID NO:2. Additionally, antigens can be generated to portions of the polypeptide which are likely to be on the surface of the folded protein. These antigens include: residue 50 to residue 67 of SEQ ID NO:2; residue 74 to residue 81 of SEQ ID NO:2; residue 161 to residue 168 of SEQ ID NO:2; residue 171 to residue 178 of SEQ ID NO:2; residue 195 to residue 205 of SEQ ID NO:2; residue 269 to residue 276 of SEQ ID NO:2; residue 286 to residue 298 of SEQ ID NO:2; and residue 367 to residue 376 of SEQ ID NO:2. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a ZNSSP11 polypeptide or a fragment thereof. The immunogenicity of a ZNSSP11 polypeptide may be increased through the use of an adjuvant, such as alum (aluminum hydroxide) or Freund's complete or incomplete adjuvant. Polypeptides useful for immunization also include fusion polypeptides, such as fusions of ZNSSP11 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Alternative techniques for generating or selecting antibodies useful herein include in vitro exposure of lymphocytes to ZNSSP11 protein or peptide, and selection of antibody display libraries in phage or similar vectors (for instance, through use of immobilized or labeled ZNSSP11 protein or peptide). Genes encoding polypeptides having potential ZNSSP11 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a substrate or enzyme, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from CLONTECH Laboratories, Inc., (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the ZNSSP11 sequences disclosed herein to identify proteins which bind to ZNSSP11. These “binding proteins” which interact with ZNSSP11 polypeptides can be used for tagging cells; for isolating homolog polypeptides by affinity purification; they can be directly or indirectly conjugated to drugs, toxins, radionuclides and the like. These binding proteins can also be used in analytical methods such as for screening expression libraries and neutralizing activity. The binding proteins can also be used for diagnostic assays for determining circulating levels of polypeptides; for detecting or quantitating soluble polypeptides as marker of underlying pathology or disease. These binding proteins can also act as ZNSSP11 “antagonists” to block ZNSSP11 anti-complementary molecule binding in vitro and in vivo. These anti-ZNSSP11 binding proteins would be useful for modulating, for example, glycoprotein and glycolipid modification, and cell-cell interactions, in general.

As used herein, the term “binding proteins” additionally includes antibodies to ZNSSP11 polypeptides, the cognate anti-complementary molecule of ZNSSP11 polypeptides, proteins useful for purification of ZNSSP11 polypeptides, and proteins associated with the catalytic domain (residues 151 to 415 of SEQ ID NO:2).

Antibodies are determined to be specifically binding if they exhibit a threshold level of binding activity (to a ZNSSP11 polypeptide, peptide or epitope) of at least 10-fold greater than the binding affinity to a control (non-ZNSSP11) polypeptide.

The binding affinity of an antibody can be readily determined by one of ordinary skill in the art, for example, by Scatchard analysis (Scatchard, G., Ann. NY Acad. Sci. 51: 660-672, 1949).

A variety of assays known to those skilled in the art can be utilized to detect antibodies which specifically bind to ZNSSP11 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmunoprecipitation, enzyme-linked immunosorbent assay (ELISA), dot blot or Western blot assay, inhibition or competition assay, and sandwich assay. In addition, antibodies can be screened for binding to wild-type versus mutant ZNSSP11 protein or polypeptide.

Antibodies to ZNSSP11 may be used for immunohistochemical tagging cells that express ZNSSP11; for isolating ZNSSP11 by affinity purification; for diagnostic assays for determining circulating levels of ZNSSP11 polypeptides; for detecting or quantitating soluble ZNSSP11 as marker of underlying pathology or disease; in analytical methods employing FACS; for screening expression libraries; for generating anti-idiotypic antibodies; and as neutralizing antibodies or as antagonists to block ZNSSP11 in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to ZNSSP11 or fragments thereof may be used in vitro to detect denatured ZNSSP11 or fragments thereof in assays, for example, Western Blots or other assays known in the art.

The soluble ZNSSP11 polypeptide (i.e., residues 33 to 378 of SEQ ID NO:2, or a portion thereof) is useful in studying the distribution of anti-complementary molecules in tissues or specific cell lineages, and to provide insight into complementary molecule/anti-complementary molecule biology. Using labeled ZNSSP11, cells expressing the anti-complementary molecule are identified by fluorescence immunocytometry or immunocytochemistry. Application may also be made of the specificity of Beta1,3 galactosyltransferases for their anti-complementary molecules.

Antibodies can be made to soluble, ZNSSP11 polypeptides which are His or FLAG™ tagged. Alternatively, such polypeptides form a fusion protein with Human Ig. In particular, antiserum containing polypeptide antibodies to His-tagged, or FLAG™-tagged soluble ZNSSP11 can be used in analysis of tissue distribution of ZNSSP11 by immunohistochemistry on human or primate tissue. These soluble ZNSSP11 polypeptides can also be used to immunize mice in order to produce monoclonal antibodies to a soluble human ZNSSP11 polypeptide. Monoclonal antibodies to a soluble human ZNSSP11 polypeptide can also be used to mimic anti-complementary molecule/complementary molecule coupling, resulting in activation or inactivation of the anti-complementary molecule/complementary molecule pair. For instance, it has been demonstrated that cross-linking anti-soluble CD40 monoclonal antibodies provides a stimulatory signal to B cells that have been sub-optimally activated with anti-IgM or LPS, and results in proliferation and immunoglobulin production. These same monoclonal antibodies act as antagonists when used in solution by blocking activation of the receptor. Monoclonal antibodies to ZNSSP11 can be used to determine the distribution, regulation and biological interaction of the ZNSSP11 and its anti-complementary molecule pair on specific cell lineages identified by tissue distribution studies. Soluble ZNSSP11 proteins or antibodies to the ZNSSP11 protein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (anti-complementary molecule or antigen, respectively, for instance). More specifically, ZNSSP11 polypeptides or anti-ZNSSP11 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, a fusion protein including only the secretory, transmembrane, stem, or catalytic domain may be suitable for directing a detectable molecule, a cytotoxic molecule or a complementary molecule to a cell or tissue type of interest. Similarly, the corresponding anti-complementary molecule to ZNSSP11 can be conjugated to a detectable or cytotoxic molecule and provide a generic targeting vehicle for cell/tissue-specific delivery of generic anti-complementary-detectable/cytotoxic molecule conjugates.

In another embodiment, ZNSSP11-cytokine fusion proteins or antibody-cytokine fusion proteins can be used for enhancing in vivo killing of target tissues (for example, tissues of diseased and/or tumorous colon, rectum, gallbladder, small intestine, and testis), if the ZNSSP11 polypeptide or anti-ZNSSP11 antibody targets hyperproliferative tissues from these organs. (See, generally, Hornick et al., Blood 89:4437-47, 1997). They described fusion proteins that enable targeting of a cytokine to a desired site of action, thereby providing an elevated local concentration of cytokine. Suitable ZNSSP11 polypeptides or anti-ZNSSP11 antibodies target an undesirable cell or tissue (i.e., a tumor or a leukemia), and the fused cytokine mediates improved target cell lysis by effector cells. Suitable cytokines for this purpose include interleukin 2 and granulocyte-macrophage colony-stimulating factor (GM-CSF), for instance.

In yet another embodiment, if the ZNSSP11 polypeptide or anti-ZNSSP11 antibody targets vascular cells or tissues, such polypeptide or antibody may be conjugated with a radionuclide, and particularly with a beta-emitting radionuclide, to reduce restenosis. Such therapeutic approach poses less danger to clinicians who administer the radioactive therapy. The bioactive polypeptide or antibody conjugates described herein can be delivered intravenously, intraarterially or intraductally, or may be introduced locally at the intended site of action.

ZNSSP11 polynucleotides and/or polypeptides may be useful for regulating the maturation of Beta1,3 galactosyltransferase anti-complementary molecule-bearing cells, such as fibroblasts, lymphocytes and hematopoietic cells. ZNSSP11 polypeptides will also find use in mediating metabolic or physiological processes in vivo. The effects of a compound on proliferation and differentiation can be measured in vitro using cultured cells. Bioassays and ELISAs are available to measure cellular response to ZNSSP11, in particular are those which measure changes in cytokine production as a measure of cellular response (see for example, Current Protocols in Immunology ed. John E. Coligan et al., NIH, 1996). Assays to measure other cellular responses, including glycoprotein and glycolipid modulations, and cell-cell interactions are known in the art.

The ZNSSP11 polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a ZNSSP11 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a ZNSSP11 polypeptide into the secretory pathway of a host cell, a secretory signal sequence (also known as a leader sequence, prepro sequence or pre sequence) is provided in the expression vector. The secretory signal sequence may be derived from another secreted protein (e.g., t-PA) or a ZNSSP11 secretory peptide (residues 1 to 14 of SEQ ID NO:2). Alternatively, it can be synthesized de novo. The secretory signal sequence is operably linked to the ZNSSP11 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

The catalytic domain of ZNSSP11 can be substituted by a heterologous sequence providing a different catalytic domain. In this case, the fusion product can be secreted, and the secretory peptide of ZNSSP11 can direct the new catalytic domain to the transmembrane and/or stem domains. This substituted catalytic domain can be chosen from the catalytic domains represented by the Beta1,3 galactosyltransferase protein families. Similarly, the catalytic domain of ZNSSP11 protein can be used to substitute the catalytic domain of a different Beta1,3 galactosyltransferase. In these cases, the fusion products can be soluble or membrane-bound proteins.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, ^{Virology} 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters can be usedsuch as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” One selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. One amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins, such as CD4, CD8, Class I MHC, or placental alkaline phosphatase, may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant ZNSSP11 baculovirus utilizes a transposon-based system described by Luckow (Luckow, V. A, et al., J Virol 67:4566-79, 1993). This system, which utilizes transfer vectors, is sold in the Bac-to-Bac™ kit (Life Technologies, Rockville, Md.). This system utilizes a transfer vector, pFastBac1™ (Life Technologies) containing a Tn7 transposon to move the DNA encoding the ZNSSP11 polypeptide into a baculovirus genome maintained in E. coli as a large plasmid called a “bacmid.” The pFastBac1™ transfer vector utilizes the AcNPV polyhedrin promoter to drive the expression of the gene of interest, in this case ZNSSP11. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol Chem 270:1543-9, 1995. In such transfer vector constructs, a short or long version of the basic protein promoter can be used. Moreover, transfer vectors can be constructed which replace the native ZNSSP11 secretory signal sequences with secretory signal sequences derived from insect proteins. For example, a secretory signal sequence from Ecdysteroid Glycosyltransferase (EGT), honey bee Melittin (Invitrogen, Carlsbad, Calif.), or baculovirus gp67 (PharMingen, San Diego, Calif.) can be used in constructs to replace the native ZNSSP11 secretory signal sequence. In addition, transfer vectors can include an in-frame fusion with DNA encoding an epitope tag at the C- or N-terminus of the expressed ZNSSP11 polypeptide, for example, a Glu-Glu epitope tag (Grussenmeyer, T. et al., Proc. Natl. Acad. Sci. 82:7952-4, 1985). Using a technique known in the art, a transfer vector containing ZNSSP11 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses ZNSSP11 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the ZNSSP11 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). One vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349.Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. The use of Pichia methanolica as host for the production of recombinant proteins is disclosed in U.S. Pat. Nos. 5,716,808, 5,736,383, 5,854,039, and 5,888,768.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a ZNSSP11 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. One culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for ZNSSP11 amino acid residues.

The polypeptides of the present invention are purified to ≧80% purity, to ≧90% purity, to ≧95% purity, and or a pharmaceutically pure state, that is greater than 99.9% pure with respect to contaminating macromolecules, particularly other proteins and nucleic acids, and free of infectious and pyrogenic agents. A purified polypeptide is substantially free of other polypeptides, particularly other polypeptides of animal origin.

Expressed recombinant ZNSSP11 proteins (including chimeric polypeptides and multimeric proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, New York, 1994. Proteins comprising a polyhistidine affinity tag (typically about 6 histidine residues) are purified by affinity chromatography on a nickel chelate resin. See, for example, Houchuli et al., Bio/Technol. 6: 1321-1325, 1988. Proteins comprising a glu-glu tag can be purified by immunoaffinity chromatography according to conventional procedures. See, for example, Grussenmeyer et al., ibid. Maltose binding protein fusions are purified on an amylose column according to methods known in the art.

The polypeptides of the present invention can be isolated by a combination of procedures including, but not limited to, anion and cation exchange chromatography, size exclusion, and affinity chromatography. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., maltose-binding protein, an immunoglobulin domain) may be constructed to facilitate purification.

ZNSSP11 polypeptides, or fragments thereof, can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

Using methods known in the art, ZNSSP11 proteins can be prepared as monomers or multimers; glycosylated or non-glycosylated; pegylated or non-pegylated; and may or may not include an initial methionine amino acid residue.

The activity of ZNSSP11 polypeptides can be measured using a variety of assays that measure, for example, cell-cell interactions, glycolipid and glycoprotein modifications, development, and other biological functions associated with Beta1,3 galactosyltransferase family members or with Beta1,3 galactosyltransferase interactions, such as, differentiation, and proliferation for example. Of particular are changes in glycoprotein and glycolipid modifications, and in cell-cell interactions in tissues of diseased and/or tumorous colon, rectum, gallbladder, small intestine, and testis, as well as normal colon and brain. Such assays are well known in the art. For a general reference, see Kolbinger, F. et al., J. Biol. Chem. 273: 433-440, 1998; Amado, M. et al., J. Biol. Chem. 273:12770-12778, 1998; Hennet, T. et al., J. Biol. Chem. 273:58-65, 1998; and Ram B. P., and Munjal, D. D., CRC Crit. Rev. Biochem. 17:257-311, 1985. Specific assays include, but are not limited to bioassays measuring cell migration, contact inhibition, tissue interactions, fertilization, embryonic cell adhesions, limb bud morphogenesis, mesenchyme development, immune recognition, growth control, tumor metastasis and suppression, glycoprotein and glycolipid modifications, and cell-cell interactions.

Additional activities likely associated with the polypeptides of the present invention include proliferation of cells of the colon and brain directly or indirectly through other growth factors; action as a chemotaxic factor; and as a factor for expanding pancreas and mesenchymal stem cell and precursor populations.

Proteins, including alternatively spliced peptides, of the present invention are useful for, for example, tumor diagnosis and staging, tumor suppression, glycoprotein and glycolipid modification, and cell-cell interactions, in general, as well as modulating growth and differentiation either working in isolation, or in conjunction with other molecules (growth factors, cytokines, etc.) in tissues of diseased and/or tumorous colon, rectum, gallbladder, small intestine, and testis, as well as normal colon and brain. Alternative splicing of ZNSSP11 may be cell-type specific and confer activity to specific anti-complementary molecules.

As polynucleotides of the present invention have been identified in tissues of diseased and/or tumorous colon, rectum, gallbladder, small intestine, and testis, polynucleotides of ZNSSP11 can be used to detect these cell types by hybridization, or with ZNSSP11 binding proteins. Similarly antibodies to the ZNSSP11 polypeptides can detect cells expressing the surface bound ZNSSP11 receptor. Such detection can be useful to determine metastasis, disease stage, or primary diagnosis of disease. Additionally, contacting the cells with ZNSSP11 binding proteins, including a ZNSSP11-cognate ligand, or an antibody to ZNSSP11, for example, can modulate proliferation, differentiation and/or apoptosis of these cell types. The proliferation, differentiation and/or apoptosis of such cells can also be mediated by ZNSSP11 polypeptides, or fragments thereof, as an antagonist of cell signaling by binding the ligand and thereby reducing the interaction between ligand and membrane-bound ZNSSP11 proteins. The effects of the modulation of ZNSSP11 molecules on proliferation, differentiation and/or apoptosis can be measured, for example, by the presence or absence of cell markers specific to these cell types, or by measuring other manifestations of these diseases. For example, ZNSSP11 polypeptides or fragments thereof, as well as ZNSSP11 binding proteins can be administered to cells of tissues of diseased and/or tumorous colon, rectum, gallbladder, small intestine, and testis, as well and inhibition of these cell types can be monitored by conventional imaging techniques.

Cancer of the large bowel is a leading cause of death in many developed countries. Most screening programs involving early detection of colorectal cancers have focused on digital rectal examinations and testing for the presence of occult blood measured by a Hemoccult test. However, approximately 50 percent of patients with documented colorectal cancers have a negative fecal Hemoccult test due to the intermittent bleeding pattern of these tumors. See Harrison's Principles of Internal Medicine, Volume 1, 14^{th} Edition, 1998, pp. 574-576. Screening tests for large-bowel cancer in asymptomatic persons remains unsatisfactory, and more effective techniques are needed. As a molecule that shows higher expression in tumorous colon and rectum than in normal colon, ZNSSP11 polypeptides, polynucleotides, fragments thereof, or associated binding proteins can be used to diagnose, and/or monitor colorectal cancer. Such diagnosis and monitoring can involve in vivo detection of the ZNSSP11 polypeptides, polynucleotides, fragments thereof, or associated binding proteins by imaging techniques known to one skilled in the art, or can involve detection of resected tissue, or detection in fecal matter. Also as discussed herein, ZNSSP11 polypeptides, polynucleotides, fragments thereof, or associated binding proteins can be used to selectively target cancerous tissues of the colon and/or rectum.

In the small intestine, ZNSSP11 expression was observed in tissue associated with chronic ulcerative colitis. Methods of detection, diagnosis, and treatment similar to those as described above for colorectal cancers can also be applied to detecting, diagnosing, and treating ulcerative colitis of the small intestine.

ZNSSP11 has also been identified in testis tumor. Methods of detection, diagnosis, and treatment similar to those as described above for colorectal cancers can also be applied to detecting, diagnosing, and treating testicular cancer.

Gallbladder disease is most often associated with the development of gall stones which cause inflammation or obstruction of the cystic duct or common bile duct. Obstruction of the cystic duct by a stone can result in acute inflammation of the duct, resulting in acute cholecystitis. The inflammation associated with acute cholecystitis can be evoked by mechanical inflammation, chemical inflammation, and bacterial inflammation. Chronic cholecystits is almost always associated with the presence of gallstones and is thought to result from repeated bouts of subacute or acute cholecystitis or from persistent mechanical irritation of the gallbladder wall. See A Harrison's Principles of Internal Medicine, Volume 1, 14^{th} Edition, 1998, pp. 1726 to 1733. As a protein that shows expression in chronic cholecystitis, ZNSSP11 polypeptides, polynucleotides, fragments thereof, or associated binding proteins can be used to detect this disease. One embodiment of the invention involves using ZNSSP11 molecules to detect and diagnose the degree or stage of inflammation of the gallbladder. Another embodiment of the invention involves using ZNSSP11 polypeptides, polynucleotides, fragments thereof, or associated binding proteins to assist in identifying inflamed gallbladder tissue during surgery.

Another assay of interest measures or detects changes in proliferation, differentiation, and development. Additionally, the effects of a ZNSSP11 polypeptides on cell-cell interactions of epithelial cells, tumor cells and diseased cells of the colon, rectum, gallbladder, small intestine, and testis, as well as normal colon and brain in particular, would be of interest to measure. Yet other assays examines changes in glycoprotein and glycolipid mocifications.

The activity of molecules of the present invention can be measured using a variety of assays that, for example, measure neogenesis or hyperplasia (i.e., proliferation) of tissues of the brain, colon, rectum, gallbladder, small intestine, and testis. Additional activities likely associated with the polypeptides of the present invention include proliferation of endothelial cells, fibroblasts, and lymphoid cells directly or indirectly through other growth factors; action as a chemotaxic factor for endothelial cells, fibroblasts and/or phagocytic cells; and factor for expanding mesenchymal stem cell and precursor populations.

Proliferation can be measured using cultured tissues of diseased and/or tumorous colon, rectum, gallbladder, small intestine, and testis, as well as normal colon and brain cells or in vivo by administering molecules of the claimed invention to an appropriate animal model. Generally, proliferative effects are observed as an increase in cell number and therefore, may include inhibition of metastasis, as well as mitogenesis. Cultured cells include colon tumor and testis tumor primary cultures. Established cell lines are easily identifiable by one skilled in the art and are available from ATCC (Manasas, Va.). Assays measuring cell proliferation are well known in the art. For example, assays measuring proliferation include such assays as chemosensitivity to neutral red dye (Cavanaugh et al., Investigational New Drugs 8:347-354, 1990), incorporation of radiolabelled nucleotides (Cook et al., Analytical Biochem. 179:1-7, 1989), incorporation of 5-bromo-2′-deoxyuridine (BrdU) in the DNA of proliferating cells (Porstmann et al., J. Immunol. Methods 82:169-179, 1985), and use of tetrazolium salts (Mosmann, J. Immunol. Methods 65:55-63, 1983; Alley et al., Cancer Res. 48:589-601, 1988; Marshall et al., Growth Reg. 5:69-84, 1995; and Scudiero et al., Cancer Res. 48:4827-4833, 1988).

To determine if ZNSSP11 is a chemotractant in vivo, ZNSSP11 can be given by intradermal or intraperitoneal injection. Characterization of the accumulated leukocytes at the site of injection can be determined using lineage specific cell surface markers and fluorescence immunocytometry or by immunohistochemistry (Jose, J. Exp. Med. 179:881-87, 1994). Release of specific leukocyte cell populations from bone marrow into peripheral blood can also be measured after ZNSSP11 injection.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products and enzymes and enzyme-like complementary molecules. The stage of a cell population's differentiation is monitored by identification of markers present in the cell population. For example, myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages. The novel polypeptides of the present invention are useful for studies to isolate mesenchymal stem cells and kidney fibroblast progenitor cells, both in vivo and ex vivo.

There is evidence to suggest that factors that stimulate specific cell types down a pathway towards terminal differentiation or dedifferentiation affect the entire cell population originating from a common precursor or stem cell. Thus, ZNSSP11 polypeptides may stimulate inhibition or proliferation of endocrine and exocrine cells of the tissues of diseased and/or tumorous colon, rectum, gallbladder, small intestine, and testis, as well as normal colon and brain.

Molecules of the present invention may, while stimulating proliferation or differentiation of fibroblasts, inhibit proliferation or differentiation of adipocytes, by virtue of their effect on common precursor/stem cells. The novel polypeptides of the present invention are useful to study neural and epithelial stem cells and brain, colon, rectum, gallbladder, small intestine, and testis in progenitor cells, both in vivo and ex vivo.

Assays measuring differentiation include, for example, measuring cell-surface markers associated with stage-specific expression of a tissue, enzymatic activity, functional activity or morphological changes (Watt, FASEB, 5:281-284, 1991;Francis, Differentiation 57:63-75, 1994; Raes, Adv. Anim. Cell Biol. Technol. Bioprocesses, 161-171, 1989).

The ZNSSP11 polypeptides of the present invention can be used to study proliferation or differentiation in brain, colon, rectum, gallbladder, small intestine, and testis. Such methods of the present invention generally comprise incubating cells derived from these tissues in the presence and absence of ZNSSP11 polypeptide, monoclonal antibody, agonist or antagonist thereof and observing changes in cell proliferation or differentiation. Cell lines from these tissues are commercially available from, for example, American Type Culture Collection (Manasas, Va.).

Proteins, including alternatively spliced peptides, and fragments, of the present invention are useful for studying cell-cell interactions, glycolipid and glycoprotein modifications, development, fertility and other biological functions associated with Beta1,3 galactosyltransferase family members. ZNSSP11 molecules, variants, and fragments can be applied in isolation, or in conjunction with other molecules (growth factors, cytokines, etc.) in brain, colon, rectum, gallbladder, small intestine, and testis.

Proteins of the present invention are useful for delivery of therapeutic agents such as, but not limited to, proteases, radionuclides, chemotherapy agents, and small molecules. Effects of these therapeutic agents can be measured in vitro using cultured cells, ex vivo on tissue slices, or in vivo by administering molecules of the claimed invention to the appropriate animal model. An alternative in vivo approach for assaying proteins of the present invention involves viral delivery systems. Exemplary viruses for this purpose include adenovirus, herpesvirus, lentivirus, vaccinia virus and adeno-associated virus (AAV). Adenovirus, a double-stranded DNA virus, is currently the best studied gene transfer vector for delivery of heterologous nucleic acid (for a review, see T. C. Becker et al., Meth. Cell Biol. 43:161-89, 1994; and J. T. Douglas and D. T. Curiel, Science & Medicine 4:44-53, 1997). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293S cells can be grown in suspension culture at relatively high cell density to produce significant amounts of protein (see Gamier et al., Cytotechnol. 15:145-55, 1994). With either protocol, an expressed, secreted heterologous protein can be repeatedly isolated from the cell culture supernatant. Within the infected 293S cell production protocol, non-secreted proteins may also be effectively obtained.

The activity of ZNSSP11 or its anti-complementary molecule, can be measured by a silicon-based biosensor microphysiometer which measures the extracellular acidification rate or proton excretion associated with such protein interactions and subsequent physiologic cellular responses. An exemplary device is the Cytosensorm Microphysiometer manufactured by Molecular Devices, Sunnyvale, Calif. A variety of cellular responses, such as cell proliferation, ion transport, energy production, inflammatory response, regulatory and enzyme or enzyme activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including ZNSSP11 proteins, their agonists, and antagonists. The microphysiometer can be used to measure responses of a ZNSSP11-responsive eukaryotic cell, compared to a control eukaryotic cell that does not respond to ZNSSP11 polypeptide. ZNSSP11-responsive eukaryotic cells comprise cells into which a polynucleotide for ZNSSP11 has been transfected creating a cell that is responsive to ZNSSP11; or cells containing endogenous ZNSSP11 polynucleotides. Differences, measured by a change in the response of cells exposed to ZNSSP11 anti-complementary molecule, relative to a control not exposed to ZNSSP11 anti-complementary molecule, directly measure the ZNSSP11-modulated cellular responses. Moreover, such ZNSSP11-modulated responses can be assayed under a variety of stimuli. The present invention provides a method of identifying agonists and antagonists of ZNSSP11 protein, comprising providing cells responsive to a ZNSSP11 polypeptide, culturing a first portion of the cells in the absence of a test compound, culturing a second portion of the cells in the presence of a test compound, and detecting a measurable change in a cellular response of the second portion of the cells as compared to the first portion of the cells. Moreover, culturing a third portion of the cells in the presence of ZNSSP11 anti-complementary molecule and the absence of a test compound provides a positive control for the ZNSSP11-responsive cells, and a control to compare the agonist activity of a test compound with that of the ZNSSP11 anti-complementary molecule. Antagonists of ZNSSP11 can be identified by exposing the cells to ZNSSP11 anti-complementary molecule in the presence and absence of the test compound, whereby a reduction in ZNSSP11-modulated activity is indicative of antagonist activity in the test compound.

Moreover, ZNSSP11 can be used to identify cells, tissues, or cell lines which respond to a ZNSSP11-modulated pathway. The microphysiometer, described above, can be used to rapidly identify cells responsive to ZNSSP11 of the present invention. Cells can be cultured in the presence or absence of ZNSSP11 polypeptide. Those cells which elicit a measurable change in extracellular acidification in the presence of ZNSSP11 polypeptides are responsive to ZNSSP11. Such cell lines, can be used to identify anti-complementary molecules, antagonists and agonists of ZNSSP11 polypeptide as described above.

In view of the tissue distribution (tissues of diseased and/or tumorous colon, rectum, gallbladder, small intestine, and testis, as well as normal colon and brain) observed for ZNSSP11 expression, agonists (including the native catalytic domain) and antagonists have enormous potential in both in vitro and in vivo applications. Compounds identified as ZNSSP11 agonists and antagonists are useful for studying cell-cell interactions, glycolipid and glycoprotein modifications, development, and other biological functions associated with Beta1,3 galactosyltransferase family members or with Beta1,3 galactosyltransferase interactions in vitro and in vivo. For example, ZNSSP11 and agonist compounds are useful as components of defined cell culture media, and may be used alone or in combination with cytokines and hormones to replace serum that is commonly used in cell culture. Agonists are thus useful in specifically promoting the growth and/or development of cells of the myeloid and lymphoid lineages in culture. Additionally, ZNSSP11 polypeptides and ZNSSP11 agonists, including small molecules are useful as a research reagent, such as for the expansion, differentiation, and/or cell-cell interactions of tissues of diseased and/or tumorous colon, rectum, gallbladder, small intestine, and testis, as well as normal colon and brain. ZNSSP11 polypeptides are added to tissue culture media for these cell types.

Compounds identified as ZNSSP11 agonists are useful for modifying the proliferation and development of target cells in vitro and in vivo. For example, agonist compounds are useful alone or in combination with cytokines and hormones as components of defined cell culture media. Agonists are thus useful in specifically mediating the growth and/or development of ZNSSP11-bearing cells in culture. Antagonists are useful as research reagents for characterizing anti-complementary molecule-complementary molecule interaction.

The invention also provides antagonists, which either bind to ZNSSP11 polypeptides or, alternatively, to a anti-complementary molecule to which ZNSSP11 polypeptides bind, thereby inhibiting or eliminating the function of ZNSSP11. Such ZNSSP11 antagonists would include antibodies; polypeptides which bind either to the ZNSSP11 polypeptide or to its anti-complementary molecule; natural or synthetic analogs of ZNSSP11 anti-complementary molecules which retain the ability to bind the anti-complementary molecule but do not result in glycoprotein or glycolipid modifications, or cell-cell interactions. Such analogs could be peptides or peptide-like compounds. Natural or synthetic small molecules which bind to ZNSSP11 polypeptides and prevent glycoprotein or glycolipid modifications, or cell-cell interactions. Also contemplated are soluble ZNSSP11 proteins. As such, ZNSSP11 antagonists would be useful as therapeutics for treating certain disorders where blocking glycosylation or anti-complementary molecule binding would be beneficial.

ZNSSP11 polypeptides may be used within diagnostic systems to detect the presence of anti-complementary molecule polypeptides. Antibodies or other agents that specifically bind to ZNSSP11 or its anti-complementary molecule may also be used to detect the presence of circulating complementary molecule or anti-complementary molecule polypeptides. Such detection methods are well known in the art and include, for example, enzyme-linked immunosorbent assay (ELISA) and radioimmunoassay. Immunohistochemically labeled ZNSSP11 antibodies can be used to detect ZNSSP11 and/or its anti-complementary molecules in tissue samples. ZNSSP11 levels can also be monitored by such methods as RT-PCR, where ZNSSP11 mRNA can be detected and quantified. The information derived from such detection methods would provide insight into the significance of ZNSSP11 polypeptides in various diseases, and as such would serve as diagnostic tools for diseases for which altered levels of ZNSSP11 are significant. Altered levels of ZNSSP11 polypeptides may be indicative of pathological conditions including, for example, cancer and disorders of the brain, colon, rectum, gallbladder, small intestine, and testis.

Antagonists are also useful as research reagents for characterizing sites of interactions between members of complement/anti-complement pairs as well as sites of cell-cell interactions. Inhibitors of ZNSSP11 activity (ZNSSP11 antagonists) include anti-ZNSSP11 antibodies and soluble ZNSSP11 polypeptides (such as residues 151 to 415 in SEQ ID NO:2), as well as other peptidic and non-peptidic agents (including ribozymes).

ZNSSP11 can also be used to identify inhibitors (antagonists) of its activity. Test compounds are added to the assays disclosed herein to identify compounds that inhibit the activity of ZNSSP11. In addition to those assays disclosed herein, samples can be tested for inhibition of ZNSSP11 activity within a variety of assays designed to measure complementary molecule/anti-complementary molecule binding or the stimulation/inhibition of ZNSSP11-dependent cellular responses. For example, ZNSSP11-responsive cell lines can be transfected with a reporter gene construct that is responsive to a ZNSSP11-modulated cellular pathway. Reporter gene constructs of this type are known in the art, and will generally comprise a DNA response element operably linked to a gene encoding an assayable protein, such as luciferase, or a metabolite, such as cyclic AMP. DNA response elements can include, but are not limited to, cyclic AMP response elements (CRE), hormone response elements (HRE), insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8):1087-94; 1990. Hormone response elements are reviewed in Beato, Cell 56:335-44; 1989. One reporter gene construct would contain a catalytic domain (i.e., residues 151 to 415 of SEQ ID NO:2) that, upon binding a Beta1,3 galactosyltransferase anti-complementary molecule, would signal intracellularly through on of the reporters listed above. Candidate compounds, solutions, mixtures or extracts are tested for the ability to inhibit the activity of ZNSSP11 on the target cells, as evidenced by a decrease in ZNSSP11 stimulation of reporter gene expression. Assays of this type will detect compounds that directly block ZNSSP11 binding to a anti-complementary molecule as well as compounds that block processes in the cellular pathway subsequent to anti-complement molecule binding. In the alternative, compounds or other samples can be tested for direct blocking of ZNSSP11 binding to a anti-complementary molecule using ZNSSP11 tagged with a detectable label (e.g., ¹²⁵I, biotin, horseradish peroxidase, FITC, and the like). Within assays of this type, the ability of a test sample to inhibit the binding of labeled ZNSSP11 to its anti-complementary molecule is indicative of inhibitory activity, which can be confirmed through secondary assays. Beta1,3 galactosyltransferases used within binding assays may be cellular Beta1,3 galactosyltransferases, soluble Beta1,3 galactosyltransferases, or isolated, immobilized Beta1,3 galactosyltransferases.

A ZNSSP11 polypeptide can also be used for purification of anti-complementary molecule. The polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing anti-complementary molecules are passed through the column one or more times to allow anti-complementary molecules to bind to the ZNSSP11 molecule polypeptide. The anti-complementary molecule is then eluted using changes in salt concentration, chaotropic agents (guanidine HCl), or pH to disrupt anti-complementary molecule-complementary molecule binding.

An assay system that uses a anti-complementary molecule-binding complementary molecule (or an antibody, one member of a complementary/anti-complementary pair or other cell-surface binding protein) or a binding fragment thereof, and a commercially available biosensor instrument (BIAcore, Pharmacia Biosensor, Piscataway, N.J.) may be advantageously employed. Such complementary molecule, antibody, member of a complement/anti-complement pair or fragment is immobilized onto the surface of a enzyme chip. Use of this instrument is disclosed by Karlsson, J. Immunol. Methods 145:229-40, 1991 and Cunningham and Wells, J. Mol. Biol. 234:554-63, 1993. A enzyme, antibody, member or fragment is covalently attached, using amine or sulfhydryl chemistry, to dextran fibers that are attached to gold film within the flow cell. A test sample is passed through the cell. If a substrate, epitope, or opposite member of the complementary/anti-complementary pair is present in the sample, it will bind to the immobilized substrate, antibody or member, respectively, causing a change in the refractive index of the medium, which is detected as a change in surface plasmon resonance of the gold film. This system allows the determination of on- and off-rates, from which binding affinity can be calculated, and assessment of.

Anti-complementary molecule binding complementary molecule polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

A “soluble protein” is a protein that is not bound to a cell membrane. Soluble proteins most commonly bind the anti-complementary molecule and lack transmembrane and cytoplasmic domains. Soluble proteins can comprise additional amino acid residues, such as affinity tags that provide for purification of the polypeptide or provide sites for attachment of the polypeptide to a anti-complementary molecule, or immunoglobulin constant region sequences. Many cell-surface proteins have naturally occurring, soluble counterparts that are produced by proteolysis or translated from alternatively spliced mRNAs. Proteins are said to be substantially free of transmembrane and intracellular polypeptide segments when they lack sufficient portions of these segments to provide membrane anchoring or signal transduction, respectively.

Soluble forms of ZNSSP11 polypeptides may act as antagonsits to or agonists of ZNSSP11 polypeptides, and would be useful to modulate the effects of ZNSSP11 in tissues of diseased and/or tumorous colon, rectum, gallbladder, small intestine, and testis, as well as normal colon and brain. The soluble of ZNSSP11 does not contain a transmembrane domain (i.e., the polypeptide of residues 1 to 41 of SEQ ID NO:2) and may act as an agonist or antagonist of ZNSSP11 activity. Since polypeptides of this nature are not anchored to the membrane, they can act at sites distant from the tissues in which they are expressed. Thus, the activity of the soluble form of ZNSSP11 polypeptides can be more wide spread than its membrane-anchored counterpart. Both isoforms would be useful in studying the effects of the present invention in vitro an in vivo.

Molecules of the present invention can be used to identify and isolate Beta1,3 galactosyltransferases, or members of complement/anti-complement pairs involved in glycoprotein and glycolipid modifications, and cell-cell interactions. For example, proteins and peptides of the present invention can be immobilized on a column and membrane preparations run over the column (Immobilized Affinity Substrate Techniques, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and anti-complementary molecule proteins can be identified.

The molecules of the present invention will be useful in modulating, for example, glycoprotein and glycolipid modification, and cell-cell interactions, in general. The polypeptides, nucleic acid and/or antibodies of the present invention can be used in treatment of disorders associated with tumor growth and metastasis, epithelial boundary maintenance, development, and fertility. The molecules of the present invention can be used to modulate cell-cell interactions including, for example, cell adhesion, and cell fusion. Additionally they can be used to modulate glycolipid and glycoprotein modifications, or cell-cell interactions in general or to treat or prevent development of pathological conditions in such diverse tissue as brain, colon, rectum, gallbladder, small intestine, and testis. In particular, certain diseases may be amenable to such diagnosis, treatment or prevention. These diseases include, but are not limited to diseases associated with defects in glycosyltransferase activities, and cell-cell interactions, in colon, rectum, testis, gall bladder, liver, and small intestine. The molecules of the present invention can be used to modulate inhibition and proliferation of tissues in the tissues of diseased and/or tumorous colon, rectum, gallbladder, small intestine, and testis, as well as normal colon and brain.

Polynucleotides encoding ZNSSP11 polypeptides are useful within gene therapy applications where it is desired to increase or inhibit ZNSSP11 activity. If a mammal has a mutated or absent ZNSSP11 gene, the ZNSSP11 gene can be introduced into the cells of the mammal. In one embodiment, a gene encoding a ZNSSP11 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a ZNSSP11 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Similarly, the ZNSSP11 polynucleotides (SEQ ID NO:1 or SEQ ID NO:3) can be used to target specific tissues such as tissues of the tissues of diseased and/or tumorous colon, rectum, gallbladder, small intestine, and testis, as well as normal colon and brain. It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Various techniques, including antisense and ribozyme methodologies, can be used to inhibit ZNSSP11 gene transcription and translation, such as to inhibit cell proliferation in vivo. Polynucleotides that are complementary to a segment of a ZNSSP11-encoding polynucleotide (e.g., a polynucleotide as set forth in SEQ ID NOs:1 or 3) are designed to bind to ZNSSP11-encoding mRNA and to inhibit translation of such mRNA. Such antisense polynucleotides are used to inhibit expression of ZNSSP11 polypeptide-encoding genes in cell culture or in a subject.

Mice engineered to express the ZNSSP11 gene, referred to as “transgenic mice,” and mice that exhibit a complete absence of ZNSSP11 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express ZNSSP11, either ubiquitously or under a tissue-specific or tissue-restricted promoter can be used to ask whether over-expression causes a phenotype. For example, over-expression of a wild-type ZNSSP11 polypeptide, polypeptide fragment or a mutant thereof may alter normal cellular processes, resulting in a phenotype that identifies a tissue in which ZNSSP11 expression is functionally relevant and may indicate a therapeutic target for the ZNSSP11, its agonists or antagonists. For example, a transgenic mouse to engineer is one that over-expresses the soluble ZNSSP11 polypeptide (approximately amino acids 1 to 483 of SEQ ID NO:2), or the membrane-bound complementary molecule, (residues 1 to 523 of SEQ ID NO:2). Moreover, such over-expression may result in a phenotype that shows similarity with human diseases. Similarly, knockout ZNSSP11 mice can be used to determine where ZNSSP11 is absolutely required in vivo. The phenotype of knockout mice is predictive of the in vivo effects of that a ZNSSP11 antagonist, such as those described herein, may have. The human ZNSSP11 cDNA can be used to isolate murine ZNSSP11 mRNA, cDNA and genomic DNA, which are subsequently used to generate knockout mice. These mice may be employed to study the ZNSSP11 gene and the protein encoded thereby in an in vivo system, and can be used as in vivo models for corresponding human diseases. Moreover, transgenic mice expression of ZNSSP11 antisense polynucleotides or ribozymes directed against ZNSSP11, described herein, can be used analogously to transgenic mice described above.

ZNSSP11 polypeptides, variants, and fragments thereof, may be useful as replacement therapy for disorders associated with Beta-1,3-galactosyltransferase, including disorders related to, for example, tumors and disease states of the brain, colon, rectum, colon tumor, gallbladder, small intestine, and testis.

A less widely appreciated determinant of tissue morphogenesis is the process of cell rearrangement: Both cell motility and cell-cell adhesion are likely to play central roles in morphogenetic cell rearrangements. Cells need to be able to rapidly break and probably simultaneously remake contacts with neighboring cells. See Gumbiner, B. M., Cell 69:385-387, 1992. As a secreted protein in tissues of the brain, colon, rectum, gallbladder, small intestine, and testis, ZNSSP11 can play a role in intercellular rearrangement in these and other tissues and cell lines.

ZNSSP11 gene may be useful to as a probe to identify humans who have a defective ZNSSP11 gene. The strong expression of ZNSSP11 in tissues of the brain, colon, rectum, gallbladder, small intestine, and testis suggests that ZNSSP11 polynucleotides or polypeptides can be used as measured as an indication of aberrant growth in these tissues. Thus, polynucleotides and polypeptides of ZNSSP11, and mutations to them, can be used a diagnostic indicators of cancer and tumor stage in these tissues.

The ZNSSP11 polypeptide is expressed in tissues of the tissues of diseased and/or tumorous colon, rectum, gallbladder, small intestine, and testis, as well as normal colon and brain. Thus, the polypeptides of the present invention are useful in studying cell adhesion and the role thereof in metastasis and may be useful in preventing metastasis, in particular metastasis in tumors of the tissues of diseased and/or tumorous colon, rectum, gallbladder, small intestine, and testis, as well as normal colon and brain. Similarly, polynucleotides and polypeptides of ZNSSP11 may be used to replace their defective counterparts in tumor or diseased tissues. Thus, ZNSSP11 polypeptide pharmaceutical compositions of the present invention may be useful in prevention or treatment of disorders associated with pathological regulation or the expansion of these tissues. The polynucleotides of the present invention may also be used in conjunction with a regulatable promoter, thus allowing the dosage of delivered protein to be regulated.

Moreover, the activity and effect of ZNSSP11 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Tumor models include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly M S, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing ZNSSP11, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., ZNSSP11, on the ability of the tumor to recruit vasculature and undergo metastasis can thus be assessed. In addition, aside from using adenovirus, the implanted cells can be transiently transfected with ZNSSP11. Moreover, purified ZNSSP11 or ZNSSP11-conditioned media can be directly injected in to this mouse model, and hence be used in this system. Use of stable ZNSSP11 transfectants as well as use of induceable promoters to activate ZNSSP11 expression in vivo are known in the art and can be used in this system to assess ZNSSP11 induction of metastasis. For general reference see, O'Reilly M S, et al. Cell 79:315-328, 1994; and Rusciano D, et al. Murine Models of Liver Metastasis. Invasion Metastasis 14:349-361, 1995.

Tn-syndrome, also called Permanent Mixed-Field Polyagglutinability, is a very rare acquired disorder affecting all hematopoietic lineages. This syndrome is characterized by the expression of the Tn and sialosyl-Tn antigens on the cell surface. The Tn antigen has been identified as an unsubstituted α-linked N-acetyl-galactosamine linked O-glycosidically to threonine or serine residues of membrane proteins. In healthy blood, this sugar is substituted by galactose and sialic acid to form a tetrasaccharide. This Tn antigen may be a result of a deficiency in β1,3,galactosyltransferase. Expression of the Tn antigen along with the sialosyl-Tn antigen and a TF antigen (characterized by a deficiency in α,2,3, sialyl-transferase) have been recognized as a cancer-associated phenomenon for many years. See Berger, E. G. et al., Transfus. Clin. Biol. 2:103-108, 1994.

Thus, the study of this syndrome has been useful in elucidating the biology of carbohydrate glycosylation disorders and the appearance of cryptantigens on the cell surface, and cancer. Highly specific and complex tumor glycan antigens are likely of great interest in studying tissue specific tumors and ZNSSP11 can be useful for studying tissues of diseased and/or tumorous colon, rectum, gallbladder, small intestine, and testis, as well as brain.

Itzkowitz, et al., looked at the expression of these cryptantigens in tissues from normal, chronic pancreatitic, and pancreatic cancer patients. The sialosyl-Tn antigen is expressed in 97% of malignant, but 0% of normal tissues. The authors suggest that normal pancreas tissue is preferentially galactosylated resulting in less silaosyl-Tn antigen. In malignant tissue, conditions favor the sialylation of Tn antigens thereby accounting for enhanced expression of sialosyl Tn over T anitgens.

In view of the observation of ZNSSP11 molecules in tissues of diseased and/or tumorous colon, rectum, gallbladder, small intestine, and testis, as well as normal colon and brain, a defect in the ZNSSP11 gene may result in defective galactosylation of cell surface carbohydrates of these cells, leading to over sialylation of the Tn antigen. Thus, ZNSSP11 polypeptides would be useful as a brain, colon, rectum, gallbladder, testis or small intestine specific β,1,3, galactosyltransferase replacement therapy for pre-cancerous and cancer tissues. To verify the presence of such activity in ZNSSP11 containing normal cell lines and tumor cell lines, such cell lines are evaluated with respect to the presence of the Tn antigen according to procedures known in the art. See, for example, Berger et al., ibid., Itzkowitz et al., ibid. and the like.

Additionally, the lack of conditions favoring proper galactosylation may result in an increase in sialosyl Tn antigens in tissues expressing ZNSSP11, which may cause an auto-immune reaction resulting in an immune attack on the brain, colon, rectum, gallbladder, small intestine, and testis. In these cases, ZNSSP11 molecules may be used to encourage proper galactosylation and limit the antigenic recognition in tissues over expressing the sialosyl Tn antigen.

Similarly, a defective ZNSSP11 gene may result in improper glycoslation of the surface carbohydrates of the tissues of tissues of diseased and/or tumorous colon, rectum, gallbladder, small intestine, and testis, as well as normal colon and brain, thus affecting cell-cell interactions and possibly cell cycle regulation. Such cases could be treated by administering polypeptides of ZNSSP11 to mammals with such a defective gene.

The present invention also provides reagents which will find use in diagnostic applications. For example, the ZNSSP11 gene, a probe comprising ZNSSP11 DNA or RNA or a subsequence thereof can be used to determine if the ZNSSP11 gene is present on chromosome 2q37 or if a mutation has occurred. Detectable chromosomal aberrations at the ZNSSP11 gene locus include, but are not limited to, aneuploidy, gene copy number changes, insertions, deletions, restriction site changes and rearrangements. These aberrations can occur within the coding sequence, within introns, or within flanking sequences, including upstream promoter and regulatory regions, and may be manifested as physical alterations within a coding sequence or changes in gene expression level.

Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

In general, these diagnostic methods comprise the steps of (a) obtaining a genetic sample from a patient; (b) incubating the genetic sample with a polynucleotide probe or primer as disclosed above, under conditions wherein the polynucleotide will hybridize to complementary polynucleotide sequence, to produce a first reaction product; and (iii) comparing the first reaction product to a control reaction product. A difference between the first reaction product and the control reaction product is indicative of a genetic abnormality in the patient. Genetic samples for use within the present invention include genomic DNA, cDNA, and RNA. The polynucleotide probe or primer can be RNA or DNA, and will comprise a portion of SEQ ID NOs:1 or 3, the complement of SEQ ID NOs:1 or 3, or an RNA equivalent thereof. Suitable assay methods in this regard include molecular genetic techniques known to those in the art, such as restriction fragment length polymorphism (RFLP) analysis, short tandem repeat (STR) analysis employing PCR techniques, ligation chain reaction (Barany, PCR Methods and Applications 1:5-16, 1991), ribonuclease protection assays, and other genetic linkage analysis techniques known in the art (Sambrook et al., ibid.; Ausubel et. al., ibid.; Marian, Chest 108:255-65, 1995). Ribonuclease protection assays (see, e.g., Ausubel et al., ibid., ch. 4) comprise the hybridization of an RNA probe to a patient RNA sample, after which the reaction product (RNA-RNA hybrid) is exposed to RNase. Hybridized regions of the RNA are protected from digestion. Within PCR assays, a patient's genetic sample is incubated with a pair of polynucleotide primers, and the region between the primers is amplified and recovered. Changes in size or amount of recovered product are indicative of mutations in the patient. Another PCR-based technique that can be employed is single strand conformational polymorphism (SSCP) analysis (Hayashi, PCR Methods and Applications 1:34-8, 1991).

Znssp11 is expressed are tissues that contract. For example contractile tissues in which Znssp11 is expressed include small intestine and colon. The effects of Znssp11 polypeptide, its antagonists and agonists, on tissue contractility can be measured in vitro using a tensiometer with or without electrical field stimulation. Such assays are known in the art and can be applied to tissue samples, such as aortic rings, vas deferens, ilium, uterine and other contractile tissue samples, as well as to organ systems, such as atria, and can be used to determine whether Znssp11 polypeptide, its agonists or antagonists, enhance or depress contractility. Molecules of the present invention are hence useful for treating dysfunction associated with contractile tissues or can be used to suppress or enhance contractility in vivo. As such, molecules of the present invention have utility in treating, gastro-intestinal disorders, infertility, in vitro fertilization, birth control, and treating impotence or other male reproductive dysfunction.

The effect of the Znssp11 polypeptides, antagonists and agonists of the present invention on contractility of tissues, including for example, testis, and gastrointestinal tissues, can be measured in a tensiometer that measures contractility and relaxation in tissues. See, Dainty et al., J. Pharmacol. 100:767, 1990; Rhee et al., Neurotox. 16: 179, 1995; Anderson, M. B., Endocrinol. 114:364-368, 1984; and Downing, S. J. and Sherwood, O. D, Endocrinol. 116:1206-1214, 1985. For example, measuring vasodilatation of aortic rings is well known in the art. Briefly, aortic rings are taken from 4 month old Sprague Dawley rats and placed in a buffer solution, such as modified Krebs solution (118.5 mM NaCl, 4.6 mM KCl, 1.2 mM MgSO₄.7H₂O, 1.2 mM KH₂PO₄, 2.5 mM CaCl₂.2H₂O, 24.8 mM NaHCO₃ and 10 mM glucose). One of skill in the art would recognize that this method can be used with other animals, such as rabbits, other rat strains, Guinea pigs, and the like. The rings are then attached to an isometric force transducer (Radnoti Inc., Monrovia, Calif.) and the data recorded with a Ponemah physiology platform (Gould Instrument systems, Inc., Valley View, Ohio) and placed in an oxygenated (95% O₂, 5% CO₂) tissue bath containing the buffer solution. The tissues are adjusted to 1 gram resting tension and allowed to stabilize for about one hour before testing. The integrity of the rings can be tested with norepinepherin (Sigma Co., St. Louis, Mo.) and Carbachol, a muscarinic acetylcholine agonist (Sigma Co.). After integrity is checked, the rings are washed three times with fresh buffer and allowed to rest for about one hour. To test a sample for vasodilatation, or relaxation of the aortic ring tissue, the rings are contracted to two grams tension and allowed to stabilize for fifteen minutes. A Znssp11 polypeptide sample is then added to 1, 2 or 3 of the 4 baths, without flushing, and tension on the rings recorded and compared to the control rings containing buffer only. Enhancement or relaxation of contractility by Znssp11 polypeptides, their agonists and antagonists is directly measured by this method, and it can be applied to other contractile tissues such as uterus, prostate, and heart.

Molecules expressed in the testis, such as Znssp11 polypeptides, may modulate hormones, hormone receptors, growth factors, or cell-cell interactions, of the reproductive cascade or be involved in spermatogenesis, or testis development, would be useful as markers for cancer of reproductive organs and as therapeutic agents for hormone-dependent cancers, by inhibiting hormone-dependent growth and/or development of tumor cells. Human reproductive system cancers such as testicular and prostate cancers are common. Moreover, receptors for steroid hormones involved in the reproductive cascade are found in human tumors and tumor cell lines (breast, prostate, endometrial, ovarian, kidney, and pancreatic tumors) (Kakar et al., Mol. Cell. Endocrinol., 106:145-49, 1994; Kakar and Jennes, Cancer Letts., 98:57-62, 1995). Thus, expression of Znssp11 in reproductive tissues suggests that polypeptides of the present invention would be useful in diagnostic methods for the detection and monitoring of reproductive cancers.

Diagnostic methods of the present invention involve the detection of Znssp11 polypeptides in the serum or tissue biopsy of a patient undergoing analysis of reproductive function or evaluation for possible reproductive cancers, e.g., testicular or prostate cancer. Such polypeptides can be detected using immunoassay techniques and antibodies, described herein, that are capable of recognizing Znssp11 polypeptide epitopes. More specifically, the present invention contemplates methods for detecting Znssp11 polypeptides comprising:

exposing a test sample potentially containing Znssp11 polypeptides to an antibody attached to a solid support, wherein said antibody binds to a first epitope of a Znssp11 polypeptide;

washing the immobilized antibody-polypeptide to remove unbound contaminants;

exposing the immobilized antibody-polypeptide to a second antibody directed to a second epitope of a Znssp11 polypeptide, wherein the second antibody is associated with a detectable label; and

detecting the detectable label. Altered levels of Znssp11 polypeptides in a test sample, such as serum, semen, urine, sweat, saliva, biopsy, and the like, can be monitored as an indication of reproductive function or of reproductive cancer or disease, when compared against a normal control.

Additional methods using probes or primers derived, for example, from the nucleotide sequences disclosed herein can also be used to detect Znssp11 expression in a patient sample, such as a blood, urine, semen, saliva, sweat, biopsy, tissue sample, or the like. For example, probes can be hybridized to tumor tissues and the hybridized complex detected by in situ hybridization. Znssp11 sequences can also be detected by PCR amplification using cDNA generated by reverse translation of sample mRNA as a template (PCR Primer A Laboratory Manual, Dieffenbach and Dveksler, eds., Cold Spring Harbor Press, 1995). When compared with a normal control, both increases or decreases of Znssp11 expression in a patient sample, relative to that of a control, can be monitored and used as an indicator or diagnostic for disease.

For pharmaceutical use, the proteins of the present invention can be administered orally, rectally, parenterally (particularly intravenous or subcutaneous), intracisternally, intraperitoneally, topically (as douches, powders, ointments, drops or transdermal patch) bucally, or as a pulmonary or nasal inhalant. Intravenous administration will be by bolus injection or infusion over a typical period of one to several hours. In general, pharmaceutical formulations will include a ZNSSP11 protein, alone, or in conjunction with a dimeric partner, in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be in the range of 0.1 to 100 μg/kg of patient weight per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years. In general, a therapeutically effective amount of ZNSSP11 is an amount sufficient to produce a clinically significant change in scar tissue formation, tumor suppression, platelet aggregation, apoptosis, myogenesis, in tissues of diseased and/or tumorous colon, rectum, gallbladder, small intestine, and testis, as well as normal colon and brain. Similarly, a therapeutically effective amount of ZNSSP11 is an amount sufficient to produce a clinically significant change in disorders associated with these tissues.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polypeptide comprising residues 151 to 415 of SEQ ID NO:2.

2. The isolated polypeptide of according to claim 1 wherein the polypeptide comprises residues 42 to 415 of SEQ ID NO:2.

3. The isolated polypeptide of according to claim 2 wherein the polypeptide comprises residues 1 to 415 of SEQ ID NO:2.

4. An isolated polypeptide selected from the group consisting of:
a) a polypeptide comprising residues 1 to 41 of SEQ ID NO:2;
b) a polypeptide comprising residues 42 to 150 of SEQ ID NO:2;
c) a polypeptide comprising residues 151 to 415 of SEQ ID NO:2; and
d) a polypeptide comprising residues 42 to 415 of SEQ ID NO:2.

5. An isolated polynucleotide encoding the polypeptide according to claim 1.

6. The isolated polynucleotide according to claim 5, wherein the polypeptide molecule is chosen from:
a) a polypeptide comprising residues 42 to 415 of SEQ ID NO:2; and
b) a polypeptide comprising residues 1 to 415 of SEQ ID NO:2.

7. An expression vector comprising the following operably linked elements:
a) a transcription promoter;
b) a DNA segment wherein the DNA segment is a polynucleotide encoding the polypeptide of claim 1; and
c) a transcription terminator.

8. The expression vector according to claim 7 wherein the DNA segment further encodes an affinity tag.

9. A cultured cell that has been transformed by the expression vector according to claim 8, wherein said cell expresses the polypeptide encoded by the DNA segment.

10. A method of producing a polypeptide comprising culturing a cell according to claim 8, whereby said cell expresses the polypeptide encoded by the DNA segment; and recovering the polypeptide.

11. The polypeptide produced by the method of claim 10.

12. A method of producing an antibody comprising the following steps in order:
inoculating an animal with a polypeptide selected from the group consisting of:
a) a polypeptide comprising residues 1 to 41 of SEQ ID NO:2;
b) a polypeptide comprising residues 42 to 150 of SEQ ID NO:2;
c) a polypeptide comprising residues 151 to 415 of SEQ ID NO:2;
d) a polypeptide comprising residues 42 to 415 of SEQ ID NO:2; and
e) a polypeptide comprising residues 1 to 415 of SEQ ID NO:2.
wherein the polypeptide elicits an immune response in the animal to produce the antibody; and isolating the antibody from the animal.

13. An antibody produced by the method of claim 12, which binds to a residues 1 to 415 of SEQ ID NO:2.

14. The antibody of claim 12, wherein the antibody is a monoclonal antibody.

15. An antibody which specifically binds to the polypeptide of claim 3.

16. A method of producing an antibody comprising the following steps in order:
inoculating an animal with an epitope bearing portion of a polypeptide;
wherein the polypeptide elicits an immune response in the animal to produce the antibody; and
isolating the antibody from the animal.

17. An antibody produced by the method of claim 16, which binds to a residues 1 to 415 of SEQ ID NO:2.

18. The antibody of claim 17, wherein the antibody is a monoclonal antibody.

19. A method of forming a reversible complementary molecule-anti-complementary molecule complex comprising;
providing an complementary molecule comprising residues 151 to 415 of SEQ ID NO:2; and
contacting the complementary molecule with a anti-complementary molecule;
whereby the complementary molecule binds the anti-complementary molecule.

20. The method of claim 19, wherein the anti-complementary molecule is selected from the group consisting of:
a) glycoproteins;
b) glycolipids; and
c) antibodies.
